(19) United States
(12) Patent Application Publication
(10) Pub. No.: US 2026/0249093 A1
(43) Pub. Date: Aug. 27, 2026

(54) DOUBLE SEQUENTIAL AND MULTIPLE VECTOR DEFIBRILLATION FOR WEARABLE CARDIOVERTER DEFIBRILLATORS

(71) Applicant: ZOLL Medical Corporation, Chelmsford, MA (US)

(72) Inventors: Matthew L. Sundermann, Pittsburgh, PA (US); Bahar Davoudi, Pittsburgh, PA (US); Steven J. Szymkiewicz, Venetia, PA (US); Kent J. Volosin, Mars, PA (US); Gary A. Freeman, Waltham, MA (US)

(73) Assignee: ZOLL Medical Corporation, Chelmsford, MA (US)

(21) Appl. No.: 18/879,359

(22) PCT Filed: Jun. 28, 2023
(86) PCT No.: PCT/US2023/069260

§ 371 Date: Dec. 27, 2024

Related U.S. Application Data
(60) Provisional application No. 63/357,332, filed on Jun. 30, 2022.

(51) Int. Cl. A61N 1/39

(52) U.S. Cl. CPC A61N 1/3904; A61N 1/3981

(57) ABSTRACT

An ambulatory non-invasive wearable defibrillator for providing therapeutic shocks to restore cardiac function includes sensing electrode(s), therapy electrodes, high-voltage circuitry connecting first and second pairs of therapy electrodes, a garment, and processing circuitry. The processing circuitry is configured to, responsive to detecting a suspected cardiac arrhythmia condition, output an alert and provide an electrical therapeutic pulse sequence to the patient. The electrical therapeutic pulse sequence includes a first multiphasic therapeutic pulse delivered at a first energy level via a first vector, and a second multiphasic therapeutic pulse delivered at a second energy level via a second vector. A timing of the electrical therapeutic pulse sequence includes a first leading edge of the first multiphasic therapeutic pulse being delivered at a first predetermined time and a second leading edge of the second multiphasic therapeutic pulse being delivered at a second predetermined time following a delay after the first predetermined time.

### CROSS-REFERENCE TO RELATED APPLICATION

This nonprovisional application claims priority to U.S. Provisional Patent Application Ser. No. 63/357,332, filed on Jun. 30, 2022, titled “DOUBLE SEQUENTIAL AND MULTIPLE VECTOR DEFIBRILLATION FOR WEARABLE CARDIOVERTER DEFIBRILLATORS,” the entirety of which is hereby incorporated by reference.

### BACKGROUND

The present disclosure relates to a wearable cardiac treatment system configured to treat cardiac arrhythmias occurring in ambulatory and/or in-hospital patients.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac treatment devices for extended periods of time. Cardiac treatment devices may provide defibrillation shocks to the patient if an abnormal cardiac rhythm is detected. The energy level of the defibrillation shocks is set to ensure that patients are effectively treated if they experience an abnormal cardiac rhythm.

### SUMMARY

In one or more examples, an ambulatory non-invasive wearable defibrillator for providing therapeutic shocks to restore cardiac function is provided. The wearable defibrillator includes at least one sensing electrode configured to monitor surface electric signals indicative of cardiac activity of a patient, a plurality of therapy electrodes, high-voltage circuitry connecting a first pair of the plurality of therapy electrodes and a second pair of the plurality of therapy electrodes, a garment configured to be worn about a torso of the patient and further configured to house the at least one sensing electrode, the plurality of therapy electrodes, and the high-voltage circuitry, and processing circuitry in communication with the at least one sensing electrode and the plurality of therapy electrodes. The processing circuitry is configured to monitor, using the at least one sensing electrode, the surface electric signals indicative of the cardiac activity of the patient. The processing circuitry is also configured to, responsive to detecting a suspected cardiac arrhythmia condition based on the cardiac activity, output an alert for the patient regarding the suspected cardiac arrhythmia condition, and on expiry of a predetermined response period during which the patient fails to provide a response to the alert, provide an electrical therapeutic pulse sequence to the patient. The electrical therapeutic pulse sequence includes a first multiphasic therapeutic pulse delivered at a first energy level via a first vector formed by the first pair of therapy electrodes, and a second multiphasic therapeutic pulse delivered at a second energy level via a second vector formed by the second pair of therapy electrodes. A timing of the electrical therapeutic pulse sequence includes a first leading edge of the first multiphasic therapeutic pulse being delivered at a first predetermined time and a second leading edge of the second multiphasic therapeutic pulse being delivered at a second predetermined time following a delay after the first predetermined time.

Implementations of the wearable defibrillator can include one or more of the following features. The wearable defibrillator further includes at least one physiological sensor in addition to the at least one sensing electrode, the at least one physiological sensor configured to monitor physiological signals indicative of one or more of cardiovibrations, pulmonary vibrations, arterial pulse information, blood oxygenation levels, or body temperature. The plurality of therapy electrodes include a plurality of non-adhesive therapeutic electrodes. The plurality of therapy electrodes include at least one adhesive therapeutic electrode. The first multiphasic therapeutic pulse is delivered at a first higher energy level, and the second multiphasic therapeutic pulse is delivered at a second lower energy level. One or both of the first multiphasic therapeutic pulse and the second multiphasic therapeutic pulse includes a truncated exponential waveform. Each of the first multiphasic therapeutic pulse and the second multiphasic therapeutic pulse includes a defibrillation shock. Each of the first multiphasic therapeutic pulse and the second multiphasic therapeutic pulse includes a cardioversion shock. The processing circuitry is configured to detect at least one of R-wave timings or T-wave timings of ECG signals of the patient using a plurality of signals provided by the at least one sensing electrode. The first multiphasic therapeutic pulse includes a pacing shock, and the second multiphasic therapeutic pulse includes at least one of a defibrillation shock or a cardioversion shock. The first multiphasic therapeutic pulse includes a first biphasic therapeutic pulse, a first triphasic therapeutic pulse, or a first quadriphasic therapeutic pulse. The second multiphasic therapeutic pulse includes a second biphasic therapeutic pulse, a second triphasic therapeutic pulse, or a second quadriphasic therapeutic pulse.

The electrical therapeutic pulse sequence further includes a third multiphasic therapeutic pulse delivered at a third energy level. The timing of the electrical therapeutic pulse sequence includes a third leading edge of the third multiphasic therapeutic pulse being delivered at a third predetermined time following a second delay after the second predetermined time. The third multiphasic therapeutic pulse is delivered via one of the first vector or the second vector. The high-voltage circuitry further connects a third pair of the plurality of therapy electrodes, and the third multiphasic therapeutic pulse is delivered via a third vector formed by the third pair of therapy electrodes.

The first vector extends from a first geometrical center of a first one of the first pair of therapy electrodes to a second geometrical center of a second one of the first pair of therapy electrodes. The second vector extends from a third geometrical center of a first one of the second pair of therapy electrodes to a fourth geometrical center of a second one of the second pair of therapy electrodes. Projections of the first and second vectors onto a transverse plane of the patient includes an angle of between about 50 to about 150 degrees. Projections of the first and second vectors onto a coronal plane of the patient includes an angle of between about 50 to about 150 degrees. Projections of the first and second vectors onto a transverse plane of the patient includes a substantially orthogonal angle. Projections of the first and second vectors onto a coronal plane of the patient includes a substantially orthogonal angle.

The first pair of therapy electrodes includes a first therapy electrode configured to be positioned on an anterior portion of the patient's torso and a second therapy electrode configured to be positioned on a posterior portion of the patient's torso. The second pair of therapy electrodes includes a third therapy electrode configured to be positioned on the anterior portion of the patient's torso and a fourth therapy electrode configured to be positioned on the posterior portion of the patient's torso. The garment includes two pockets configured to be positioned against the anterior portion of the patient's torso and configured to receive the first and third therapy electrodes, and two pockets configured to be positioned against the posterior portion of the patient's torso configured to receive the second and fourth therapy electrodes. The first pair of therapy electrodes includes a first therapy electrode configured to be positioned on a first anterior portion of the patient's torso and a second therapy electrode configured to be positioned on a second anterior portion of the patient's torso, superior to the first therapy electrode. The second pair of therapy electrodes includes a third therapy electrode configured to be positioned on a third anterior portion of the patient's torso and a fourth therapy electrode configured to be positioned on a fourth anterior portion of the patient's torso, superior to the third therapy electrode. The garment includes four pockets configured to be positioned against the first, second, third, and fourth anterior positions of the patient's torso and receive the first, second, third, and fourth therapy electrodes.

The first energy level is less than 100 J, and the second energy level is less than 100 J. A combined energy level delivered by the first multiphasic therapeutic pulse and the second multiphasic therapeutic pulse is less than 100 J. The combined energy level is less than 90 J. The combined energy level is less than 80 J. The first energy level is at least 5 J greater than the second energy level. The first energy level is at least 10 J greater than the second energy level.

The electrical therapeutic pulse sequence is provided within 20 to 40 seconds of detecting the suspected cardiac arrhythmia condition. The electrical therapeutic pulse sequence is provided within 10 to 20 seconds of detecting the suspected cardiac arrhythmia condition. The electrical therapeutic pulse sequence is provided within 5 to 10 seconds of detecting the suspected cardiac arrhythmia condition.

The first energy level and the second energy level include default energy levels. The processing circuitry is further configured to calculate the second energy level following the first predetermined time. The processing circuitry is further configured to receive, via a user interface, an energy level user input relating to at least one of the first energy level or the second energy level, and set the at least one of the first energy level or the second energy level based on the energy level user input.

The first multiphasic therapeutic pulse includes a waveform lasting between 10 ms and 50 ms. The second multiphasic therapeutic pulse includes a waveform lasting between 10 ms and 50 ms. The delay is between 0 ms and 250 ms. The delay is between 0 ms and 50 ms. The delay is between 120 ms and 150 ms. The processing circuitry is further configured to receive, via a user interface, a delay user input providing the delay, and set the delay based on the delay user input. The delay is greater than a length of the first multiphasic therapeutic pulse. The delay is less than or equal to a length of the first multiphasic therapeutic pulse.

The processing circuitry is further configured to continue to monitor the surface electric signals indicative of cardiac activity of the patient, and determine, based on the continued monitoring, whether the patient's cardiac rhythm returned to normal after the provided electrical therapeutic pulse sequence. The processing circuitry is further configured to provide a second electrical therapeutic pulse sequence to the patient on determining that the patient's cardiac rhythm has not returned to normal. The second electrical therapeutic pulse sequence includes a third multiphasic therapeutic pulse delivered at a third energy level, and a fourth multiphasic therapeutic pulse delivered at a fourth energy level. A timing of the second electrical therapeutic pulse sequences includes a third leading edge of the third multiphasic therapeutic pulse being delivered at a third predetermined time and a fourth leading edge of the fourth multiphasic therapeutic pulse being delivered at a fourth predetermined time following a second delay after the third predetermined time. The third multiphasic therapeutic pulse is delivered via one of the first pair of therapy electrodes or the second pair of therapy electrodes, and the fourth multiphasic therapeutic pulse is delivered via the other of the first pair of therapy electrodes or the second pair of therapy electrodes. The high-voltage circuitry further connects a third pair of the plurality of therapy electrodes and a fourth pair of the plurality of therapy electrodes. The third multiphasic therapeutic pulse is delivered via a third vector formed by the third pair of therapy electrodes, and the fourth multiphasic therapeutic pulse is delivered via a fourth vector formed by the fourth pair of therapy electrodes. The third energy level is higher than the first energy level and/or the fourth energy level is higher than the second energy level. A first combined energy of the electrical therapeutic pulse sequence is less than 80 J, and wherein a second combined energy of the second electrical therapeutic pulse sequence is less than 100 J. The delay of the electrical therapeutic pulse sequence differs from the second delay of the second electrical therapeutic pulse sequence. A first combined energy of the electrical therapeutic pulse sequence differs from a second combined energy of the second electrical therapeutic pulse sequence. A first energy level delivery distribution of the electrical therapeutic pulse sequence differs from a second energy level delivery distribution of the second electrical therapeutic pulse sequence. The processing circuitry is further configured to determine whether the patient's cardiac rhythm returned to normal after the provided second electrical therapeutic pulse sequence, and adjust energy levels for a future electrical therapeutic pulse sequence based on the third energy level and the fourth energy level on determining that the patient's cardiac rhythm returned to normal.

The processing circuitry is further configured to determine at least one impedance measurement for the patient based on the provided electrical therapeutic pulse sequence, and adjust energy levels for a future electrical therapeutic pulse sequence based on the at least one impedance measurement. The processing circuitry is further configured to determine at least one intermediate impedance measurement for the patient based on the delivered first multiphasic therapeutic pulse sequence, and before delivering the second multiphasic therapeutic pulse, adjust the second energy level based on the at least one intermediate impedance measurement.

The processing circuitry is further configured to determine that the suspected cardiac arrhythmia condition includes ventricular fibrillation, and adjust at least one parameter of the electrical therapeutic pulse sequence based on the determination that the suspected cardiac arrhythmia condition includes ventricular fibrillation. The processing circuitry is further configured to determine that the suspected cardiac arrhythmia condition includes ventricular tachycardia, and adjust at least one parameter of the electrical therapeutic pulse sequence based on the determination that the suspected cardiac arrhythmia condition includes ventricular tachycardia. The at least one parameter of the electrical therapeutic pulse sequence includes at least one of the first predetermined time, the delay, the first energy level, or the second energy level.

The high-voltage circuitry includes a first high-voltage circuit connecting the first pair of the plurality of therapy electrodes, and a second high-voltage circuit connecting the second pair of the plurality of therapy electrodes. The first multiphasic therapeutic pulse is delivered via the first high-voltage circuit, and the second multiphasic therapeutic pulse is delivered via the second high-voltage circuit. The second high-voltage circuit is configured to be electrically isolated from the first high-voltage circuit. The wearable defibrillator further includes a capacitor configured to be selectively connected to the first high-voltage circuit or the second high-voltage circuit. The wearable defibrillator further includes first capacitor electrically connected to the first high-voltage circuit and a second capacitor electrically connected to the second high-voltage circuit. The first pair of therapy electrodes includes two electrically coupled electrodes of the plurality of therapy electrodes functioning as a single electrode, and the two electrically coupled electrodes are paired with another electrode of the plurality of therapy electrodes to form the first pair of therapy electrodes.

In one or more examples, a method for providing therapeutic shocks to restore cardiac function to a patient wearing an ambulatory non-invasive wearable defibrillator is implemented. The method includes monitoring, by processing circuitry of the wearable defibrillator, surface electric signals indicative of cardiac activity of the patient. The surface electric signals are generated by at least one sensing electrode of the wearable defibrillator. The wearable defibrillator includes a garment configured to be worn about a torso of the patient and further configured to house the at least one sensing electrode. The method further includes, responsive to detecting a suspected cardiac arrhythmia condition based on the cardiac activity, outputting an alert for the patient regarding the suspected cardiac arrhythmia condition, and on expiry of a predetermined response period during which the patient fails to provide a response to the alert, providing an electrical therapeutic pulse sequence to the patient. The electrical therapeutic pulse sequences includes a first multiphasic therapeutic pulse delivered at a first energy level via a first vector formed by a first pair of a plurality of therapy electrodes of the wearable defibrillator, and a second multiphasic therapeutic pulse delivered at a second energy level via a second vector formed by a second pair of the plurality of therapy electrodes. The plurality of therapy electrodes are configured to be housed by the garment and the wearable defibrillator further includes high-voltage circuitry connecting the first pair of therapy electrodes and the second pair of therapy electrodes. A timing of the electrical therapeutic pulse sequence includes a first leading edge of the first multiphasic therapeutic pulse being delivered at a first predetermined time and a second leading edge of the second multiphasic therapeutic pulse being delivered at a second predetermined time following a delay after the first predetermined time.

Implementations of the method for providing therapeutic shocks to restore cardiac function can include one or more of the following features. The method further includes monitoring, by at least one physiological sensor in addition to the at least one sensing electrode, physiological signals indicative of one or more of cardiovibrations, pulmonary vibrations, arterial pulse information, blood oxygenation levels, or body temperature. The plurality of therapy electrodes include a plurality of non-adhesive therapeutic electrodes. The plurality of therapy electrodes include at least one adhesive therapeutic electrode. The first multiphasic therapeutic pulse is delivered at a first higher energy level, and the second multiphasic therapeutic pulse is delivered at a second lower energy level. One or both of the first multiphasic therapeutic pulse and the second multiphasic therapeutic pulse includes a truncated exponential waveform. Each of the first multiphasic therapeutic pulse and the second multiphasic therapeutic pulse includes a defibrillation shock. Each of the first multiphasic therapeutic pulse and the second multiphasic therapeutic pulse includes a cardioversion shock. The method further includes detecting at least one of R-wave timings or T-wave timings of ECG signals of the patient using a plurality of signals provided by the at least one sensing electrode. The first multiphasic therapeutic pulse includes a pacing shock, and the second multiphasic therapeutic pulse includes at least one of a defibrillation shock or a cardioversion shock. The first multiphasic therapeutic pulse includes a first biphasic therapeutic pulse, a first triphasic therapeutic pulse, or a first quadriphasic therapeutic pulse. The second multiphasic therapeutic pulse includes a second biphasic therapeutic pulse, a second triphasic therapeutic pulse, or a second quadriphasic therapeutic pulse.

The electrical therapeutic pulse sequence further includes a third multiphasic therapeutic pulse delivered at a third energy level. The timing of the electrical therapeutic pulse sequence includes a third leading edge of the third multiphasic therapeutic pulse being delivered at a third predetermined time following a second delay after the second predetermined time. The third multiphasic therapeutic pulse is delivered via one of the first vector or the second vector. The third multiphasic therapeutic pulse is delivered via a third vector formed by a third pair of the plurality of therapy electrodes.

The first vector extends from a first geometrical center of a first one of the first pair of therapy electrodes to a second geometrical center of a second one of the first pair of therapy electrodes. The second vector extends from a third geometrical center of a first one of the second pair of therapy electrodes to a fourth geometrical center of a second one of the second pair of therapy electrodes. Projections of the first and second vectors onto a transverse plane of the patient includes an angle of between about 50 to about 150 degrees. Projections of the first and second vectors onto a coronal plane of the patient includes an angle of between about 50 to about 150 degrees. Projections of the first and second vectors onto a transverse plane of the patient includes a substantially orthogonal angle. Projections of the first and second vectors onto a coronal plane of the patient includes a substantially orthogonal angle.

The first pair of therapy electrodes includes a first therapy electrode configured to be positioned on an anterior portion of the patient's torso and a second therapy electrode configured to be positioned on a posterior portion of the patient's torso. The second pair of therapy electrodes includes a third therapy electrode configured to be positioned on the anterior portion of the patient's torso and a fourth therapy electrode configured to be positioned on the posterior portion of the patient's torso. The garment includes two pockets configured to be positioned against the anterior portion of the patient's torso and configured to receive the first and third therapy electrodes, and two pockets configured to be positioned against the posterior portion of the patient's torso configured to receive the second and fourth therapy electrodes. The first pair of therapy electrodes includes a first therapy electrode configured to be positioned on a first anterior portion of the patient's torso and a second therapy electrode configured to be positioned on a second anterior portion of the patient's torso, superior to the first therapy electrode. The second pair of therapy electrodes includes a third therapy electrode configured to be positioned on a third anterior portion of the patient's torso and a fourth therapy electrode configured to be positioned on a fourth anterior portion of the patient's torso, superior to the third therapy electrode. The garment includes four pockets configured to be positioned against the first, second, third, and fourth anterior positions of the patient's torso and receive the first, second, third, and fourth therapy electrodes.

The first energy level is less than 100 J, and the second energy level is less than 100 J. A combined energy level delivered by the first multiphasic therapeutic pulse and the second multiphasic therapeutic pulse is less than 100 J. The combined energy level is less than 90 J. The combined energy level is less than 80 J. The first energy level is at least 5 J greater than the second energy level. The first energy level is at least 10 J greater than the second energy level.

The electrical therapeutic pulse sequence is provided within 20 to 40 seconds of detecting the suspected cardiac arrhythmia condition. The electrical therapeutic pulse sequence is provided within 10 to 20 seconds of detecting the suspected cardiac arrhythmia condition. The electrical therapeutic pulse sequence is provided within 5 to 10 seconds of detecting the suspected cardiac arrhythmia condition.

The first energy level and the second energy level include default energy levels. The method further includes calculating the second energy level following the first predetermined time. The method further includes receiving, via a user interface, an energy level user input relating to at least one of the first energy level or the second energy level, and setting the at least one of the first energy level or the second energy level based on the energy level user input.

The first multiphasic therapeutic pulse includes a waveform lasting between 10 ms and 50 ms. The second multiphasic therapeutic pulse includes a waveform lasting between 10 ms and 50 ms. The delay is between 0 ms and 250 ms. The delay is between 0 ms and 50 ms. The delay is between 120 ms and 150 ms. The method further includes receiving, via a user interface, a delay user input providing the delay, and setting the delay based on the delay user input. The delay is greater than a length of the first multiphasic therapeutic pulse. The delay is less than or equal to a length of the first multiphasic therapeutic pulse.

The method further includes continuing to monitor the surface electric signals indicative of cardiac activity of the patient, and determining, based on the continued monitoring, whether the patient's cardiac rhythm returned to normal after the provided electrical therapeutic pulse sequence. The method further includes providing a second electrical therapeutic pulse sequence to the patient on determining that the patient's cardiac rhythm has not returned to normal. The second electrical therapeutic pulse sequence includes a third multiphasic therapeutic pulse delivered at a third energy level, and a fourth multiphasic therapeutic pulse delivered at a fourth energy level. A timing of the second electrical therapeutic pulse sequences includes a third leading edge of the third multiphasic therapeutic pulse being delivered at a third predetermined time and a fourth leading edge of the fourth multiphasic therapeutic pulse being delivered at a fourth predetermined time following a second delay after the third predetermined time. The third multiphasic therapeutic pulse is delivered via one of the first pair of therapy electrodes or the second pair of therapy electrodes, and the fourth multiphasic therapeutic pulse is delivered via the other of the first pair of therapy electrodes or the second pair of therapy electrodes. The third multiphasic therapeutic pulse is delivered via a third vector formed by a third pair of the plurality of therapy electrodes, and the fourth multiphasic therapeutic pulse is delivered via a fourth vector formed by a fourth pair of the plurality of therapy electrodes. The third energy level is higher than the first energy level and/or the fourth energy level is higher than the second energy level. A first combined energy of the electrical therapeutic pulse sequence is less than 80 J, and a second combined energy of the second electrical therapeutic pulse sequence is less than 100 J. The delay of the electrical therapeutic pulse sequence differs from the second delay of the second electrical therapeutic pulse sequence. A first combined energy of the electrical therapeutic pulse sequence differs from a second combined energy of the second electrical therapeutic pulse sequence. A first energy level delivery distribution of the electrical therapeutic pulse sequence differs from a second energy level delivery distribution of the second electrical therapeutic pulse sequence. The method further includes determining whether the patient's cardiac rhythm returned to normal after the provided second electrical therapeutic pulse sequence, and adjusting energy levels for a future electrical therapeutic pulse sequence based on the third energy level and the fourth energy level on determining that the patient's cardiac rhythm returned to normal.

The method further includes determining at least one impedance measurement for the patient based on the provided electrical therapeutic pulse sequence, and adjusting energy levels for a future electrical therapeutic pulse sequence based on the at least one impedance measurement. The method further includes determining at least one intermediate impedance measurement for the patient based on the delivered first multiphasic therapeutic pulse sequence, and before delivering the second multiphasic therapeutic pulse, adjusting the second energy level based on the at least one intermediate impedance measurement.

The method further includes determining that the suspected cardiac arrhythmia condition includes ventricular fibrillation, and adjusting at least one parameter of the electrical therapeutic pulse sequence based on the determination that the suspected cardiac arrhythmia condition includes ventricular fibrillation. The method further includes determining that the suspected cardiac arrhythmia condition includes ventricular tachycardia, and adjusting at least one parameter of the electrical therapeutic pulse sequence based on the determination that the suspected cardiac arrhythmia condition includes ventricular tachycardia. The at least one parameter of the electrical therapeutic pulse sequence includes at least one of the first predetermined time, the delay, the first energy level, or the second energy level.

The wearable defibrillator further includes a first high-voltage circuit connecting the first pair of the plurality of therapy electrodes, and a second high-voltage circuit connecting the second pair of the plurality of therapy electrodes. The first multiphasic therapeutic pulse is delivered via the first high-voltage circuit, and the second multiphasic therapeutic pulse is delivered via the second high-voltage circuit. The second high-voltage circuit is configured to be electrically isolated from the first high-voltage circuit. The wearable defibrillator further includes a capacitor configured to be selectively connected to the first high-voltage circuit or the second high-voltage circuit. The wearable defibrillator further includes first capacitor electrically connected to the first high-voltage circuit and a second capacitor electrically connected to the second high-voltage circuit. The first pair of therapy electrodes includes two electrically coupled electrodes of the plurality of therapy electrodes functioning as a single electrode, and the two electrically coupled electrodes are paired with another electrode of the plurality of therapy electrodes to form the first pair of therapy electrodes.

In one or more examples, a non-transitory computer-readable medium storing sequences of instructions executable by at least one processor is provided. The sequences of instructions instruct the at least one processor to provide therapeutic shocks to restore cardiac function to a patient wearing an ambulatory non-invasive wearable defibrillator. The sequences of instructions include instructions to monitor, by processing circuitry of the wearable defibrillator, surface electric signals indicative of cardiac activity of the patient. The surface electric signals are generated by at least one sensing electrode of the wearable defibrillator. The wearable defibrillator includes a garment configured to be worn about a torso of the patient and further configured to house the at least one sensing electrode. The sequences of instructions further include instructions to, responsive to detecting a suspected cardiac arrhythmia condition based on the cardiac activity, output an alert for the patient regarding the suspected cardiac arrhythmia condition, and on expiry of a predetermined response period during which the patient fails to provide a response to the alert, provide an electrical therapeutic pulse sequence to the patient. The electrical therapeutic pulse sequence includes a first multiphasic therapeutic pulse delivered at a first energy level via a first vector formed by a first pair of a plurality of therapy electrodes of the wearable defibrillator, and a second multiphasic therapeutic pulse delivered at a second energy level via a second vector formed by a second pair of the plurality of therapy electrodes. The plurality of therapy electrodes are configured to be housed by the garment and the wearable defibrillator further includes high-voltage circuitry connecting the first pair of therapy electrodes and the second pair of therapy electrodes. A timing of the electrical therapeutic pulse sequence includes a first leading edge of the first multiphasic therapeutic pulse being delivered at a first predetermined time and a second leading edge of the second multiphasic therapeutic pulse being delivered at a second predetermined time following a delay after the first predetermined time.

Implementations of the non-transitory computer-readable medium can include one or more of the following features. The sequences of instructions further include instructions to monitor, by at least one physiological sensor in addition to the at least one sensing electrode, physiological signals indicative of one or more of cardiovibrations, pulmonary vibrations, arterial pulse information, blood oxygenation levels, or body temperature. The plurality of therapy electrodes include a plurality of non-adhesive therapeutic electrodes. The plurality of therapy electrodes include at least one adhesive therapeutic electrode. The first multiphasic therapeutic pulse is delivered at a first higher energy level, and the second multiphasic therapeutic pulse is delivered at a second lower energy level. One or both of the first multiphasic therapeutic pulse and the second multiphasic therapeutic pulse includes a truncated exponential waveform. Each of the first multiphasic therapeutic pulse and the second multiphasic therapeutic pulse includes a defibrillation shock. Each of the first multiphasic therapeutic pulse and the second multiphasic therapeutic pulse includes a cardioversion shock. The sequences of instructions further include instructions to detect at least one of R-wave timings or T-wave timings of ECG signals of the patient using a plurality of signals provided by the at least one sensing electrode. The first multiphasic therapeutic pulse includes a pacing shock, and the second multiphasic therapeutic pulse includes at least one of a defibrillation shock or a cardioversion shock. The first multiphasic therapeutic pulse includes a first biphasic therapeutic pulse, a first triphasic therapeutic pulse, or a first quadriphasic therapeutic pulse. The second multiphasic therapeutic pulse includes a second biphasic therapeutic pulse, a second triphasic therapeutic pulse, or a second quadriphasic therapeutic pulse.

The electrical therapeutic pulse sequence further includes a third multiphasic therapeutic pulse delivered at a third energy level. The timing of the electrical therapeutic pulse sequence includes a third leading edge of the third multiphasic therapeutic pulse being delivered at a third predetermined time following a second delay after the second predetermined time. The third multiphasic therapeutic pulse is delivered via one of the first vector or the second vector. The third multiphasic therapeutic pulse is delivered via a third vector formed by a third pair of the plurality of therapy electrodes.

The first vector extends from a first geometrical center of a first one of the first pair of therapy electrodes to a second geometrical center of a second one of the first pair of therapy electrodes. The second vector extends from a third geometrical center of a first one of the second pair of therapy electrodes to a fourth geometrical center of a second one of the second pair of therapy electrodes. Projections of the first and second vectors onto a transverse plane of the patient includes an angle of between about 50 to about 150 degrees. Projections of the first and second vectors onto a coronal plane of the patient includes an angle of between about 50 to about 150 degrees. Projections of the first and second vectors onto a transverse plane of the patient includes a substantially orthogonal angle. Projections of the first and second vectors onto a coronal plane of the patient includes a substantially orthogonal angle.

The first pair of therapy electrodes includes a first therapy electrode configured to be positioned on an anterior portion of the patient's torso and a second therapy electrode configured to be positioned on a posterior portion of the patient's torso. The second pair of therapy electrodes includes a third therapy electrode configured to be positioned on the anterior portion of the patient's torso and a fourth therapy electrode configured to be positioned on the posterior portion of the patient's torso. The garment includes two pockets configured to be positioned against the anterior portion of the patient's torso and configured to receive the first and third therapy electrodes, and two pockets configured to be positioned against the posterior portion of the patient's torso configured to receive the second and fourth therapy electrodes. The first pair of therapy electrodes includes a first therapy electrode configured to be positioned on a first anterior portion of the patient's torso and a second therapy electrode configured to be positioned on a second anterior portion of the patient's torso, superior to the first therapy electrode. The second pair of therapy electrodes includes a third therapy electrode configured to be positioned on a third anterior portion of the patient's torso and a fourth therapy electrode configured to be positioned on a fourth anterior portion of the patient's torso, superior to the third therapy electrode. The garment includes four pockets configured to be positioned against the first, second, third, and fourth anterior positions of the patient's torso and receive the first, second, third, and fourth therapy electrodes.

The first energy level is less than 100 J, and the second energy level is less than 100 J. A combined energy level delivered by the first multiphasic therapeutic pulse and the second multiphasic therapeutic pulse is less than 100 J. The combined energy level is less than 90 J. The combined energy level is less than 80 J. The first energy level is at least 5 J greater than the second energy level. The first energy level is at least 10 J greater than the second energy level.

The electrical therapeutic pulse sequence is provided within 20 to 40 seconds of detecting the suspected cardiac arrhythmia condition. The electrical therapeutic pulse sequence is provided within 10 to 20 seconds of detecting the suspected cardiac arrhythmia condition. The electrical therapeutic pulse sequence is provided within 5 to 10 seconds of detecting the suspected cardiac arrhythmia condition.

The first energy level and the second energy level include default energy levels. The sequences of instructions further include instructions to calculate the second energy level following the first predetermined time. The sequences of instructions further include instructions to receive, via a user interface, an energy level user input relating to at least one of the first energy level or the second energy level, and set the at least one of the first energy level or the second energy level based on the energy level user input.

The first multiphasic therapeutic pulse includes a waveform lasting between 10 ms and 50 ms. The second multiphasic therapeutic pulse includes a waveform lasting between 10 ms and 50 ms. The delay is between 0 ms and 250 ms. The delay is between 0 ms and 50 ms. The delay is between 120 ms and 150 ms. The method further includes receiving, via a user interface, a delay user input providing the delay, and setting the delay based on the delay user input. The delay is greater than a length of the first multiphasic therapeutic pulse. The delay is less than or equal to a length of the first multiphasic therapeutic pulse.

The sequences of instructions further include instructions to continue to monitor the surface electric signals indicative of cardiac activity of the patient, and determine, based on the continued monitoring, whether the patient's cardiac rhythm returned to normal after the provided electrical therapeutic pulse sequence. The sequences of instructions further include instructions to provide a second electrical therapeutic pulse sequence to the patient on determining that the patient's cardiac rhythm has not returned to normal. The second electrical therapeutic pulse sequence includes a third multiphasic therapeutic pulse delivered at a third energy level, and a fourth multiphasic therapeutic pulse delivered at a fourth energy level. A timing of the second electrical therapeutic pulse sequences includes a third leading edge of the third multiphasic therapeutic pulse being delivered at a third predetermined time and a fourth leading edge of the fourth multiphasic therapeutic pulse being delivered at a fourth predetermined time following a second delay after the third predetermined time. The third multiphasic therapeutic pulse is delivered via one of the first pair of therapy electrodes or the second pair of therapy electrodes, and the fourth multiphasic therapeutic pulse is delivered via the other of the first pair of therapy electrodes or the second pair of therapy electrodes. The third multiphasic therapeutic pulse is delivered via a third vector formed by a third pair of the plurality of therapy electrodes, and the fourth multiphasic therapeutic pulse is delivered via a fourth vector formed by a fourth pair of the plurality of therapy electrodes. The third energy level is higher than the first energy level and/or the fourth energy level is higher than the second energy level. A first combined energy of the electrical therapeutic pulse sequence is less than 80 J, and a second combined energy of the second electrical therapeutic pulse sequence is less than 100 J. The delay of the electrical therapeutic pulse sequence differs from the second delay of the second electrical therapeutic pulse sequence. A first combined energy of the electrical therapeutic pulse sequence differs from a second combined energy of the second electrical therapeutic pulse sequence. A first energy level delivery distribution of the electrical therapeutic pulse sequence differs from a second energy level delivery distribution of the second electrical therapeutic pulse sequence. The sequences of instructions further include instructions to determine whether the patient's cardiac rhythm returned to normal after the provided second electrical therapeutic pulse sequence, and adjust energy levels for a future electrical therapeutic pulse sequence based on the third energy level and the fourth energy level on determining that the patient's cardiac rhythm returned to normal.

The sequences of instructions further include instructions to determine at least one impedance measurement for the patient based on the provided electrical therapeutic pulse sequence, and adjust energy levels for a future electrical therapeutic pulse sequence based on the at least one impedance measurement. The sequences of instructions further include instructions to determine at least one intermediate impedance measurement for the patient based on the delivered first multiphasic therapeutic pulse sequence, and before delivering the second multiphasic therapeutic pulse, adjust the second energy level based on the at least one intermediate impedance measurement.

The sequences of instructions further include instructions to determine that the suspected cardiac arrhythmia condition includes ventricular fibrillation, and adjust at least one parameter of the electrical therapeutic pulse sequence based on the determination that the suspected cardiac arrhythmia condition includes ventricular fibrillation. The sequences of instructions further include instructions to determine that the suspected cardiac arrhythmia condition includes ventricular tachycardia, and adjust at least one parameter of the electrical therapeutic pulse sequence based on the determination that the suspected cardiac arrhythmia condition includes ventricular tachycardia. The at least one parameter of the electrical therapeutic pulse sequence includes at least one of the first predetermined time, the delay, the first energy level, or the second energy level.

The wearable defibrillator further includes a first high-voltage circuit connecting the first pair of the plurality of therapy electrodes, and a second high-voltage circuit connecting the second pair of the plurality of therapy electrodes. The first multiphasic therapeutic pulse is delivered via the first high-voltage circuit, and the second multiphasic therapeutic pulse is delivered via the second high-voltage circuit. The second high-voltage circuit is configured to be electrically isolated from the first high-voltage circuit. The wearable defibrillator further includes a capacitor configured to be selectively connected to the first high-voltage circuit or the second high-voltage circuit. The wearable defibrillator further includes first capacitor electrically connected to the first high-voltage circuit and a second capacitor electrically connected to the second high-voltage circuit. The first pair of therapy electrodes includes two electrically coupled electrodes of the plurality of therapy electrodes functioning as a single electrode, and the two electrically coupled electrodes are paired with another electrode of the plurality of therapy electrodes to form the first pair of therapy electrodes.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example treatment sequence for a patient wearing a wearable defibrillator.

FIG. 2 depicts an example wearable cardiac treatment system including a wearable defibrillator.

FIG. 3 depicts an example wearable defibrillator.

FIG. 4 depicts an example process flow for detecting and treating a suspected cardiac arrhythmia condition.

FIG. 5 depicts an example process flow for determining whether a patient is experiencing a suspected cardiac arrhythmia condition.

FIG. 6 depicts an example process flow for providing an electrical therapeutic pulse sequence to a patient.

FIG. 7A depicts an example biphasic waveform.

FIG. 7B depicts another example biphasic waveform.

FIG. 7C depicts another example biphasic waveform.

FIG. 7D depicts another example biphasic waveform.

FIG. 7E depicts another example biphasic waveform.

FIG. 7F depicts another example biphasic waveform.

FIG. 7G depicts an example triphasic waveform.

FIG. 8A depicts an example placement for therapy electrodes of a wearable defibrillator.

FIG. 8B depicts another example placement for therapy electrodes of a wearable defibrillator.

FIG. 8C depicts another example placement for therapy electrodes of a wearable defibrillator.

FIG. 8D depicts another example placement for therapy electrodes of a wearable defibrillator.

FIG. 8E depicts another example placement for therapy electrodes of a wearable defibrillator.

FIG. 8F depicts another example placement for therapy electrodes of a wearable defibrillator.

FIG. 8G depicts another example placement for therapy electrodes of a wearable defibrillator.

FIG. 8H depicts another example placement for therapy electrodes of a wearable defibrillator.

FIG. 9A depicts an example electronic architecture for a wearable defibrillator.

FIG. 9B depicts another example electronic architecture for a wearable defibrillator.

FIG. 9C depicts example circuitry for generating and delivering therapeutic pulses in accordance with this disclosure.

FIG. 10 depicts another example wearable defibrillator.

FIG. 11 depicts another example wearable defibrillator.

FIG. 12 depicts another example wearable defibrillator.

FIG. 13 depicts an example of vectors that can be created between therapy electrodes.

FIG. 14 depicts another example of vectors that can be created between therapy electrodes.

FIG. 15 depicts an example of multiple therapeutic waveforms that can be delivered to a patient.

FIG. 16 depicts another example of multiple therapeutic waveforms that can be delivered to a patient.

### DETAILED DESCRIPTION

Wearable medical devices, such as wearable cardiac treatment devices, are used in clinical, outpatient, or in-hospital (inpatient) care settings to monitor for treatable cardiac arrhythmias, alert about such arrhythmias, and automatically provide treatment such as defibrillation, cardioversion, or pacing shocks in the event of life-threatening arrhythmias. In examples, clinical settings include a broad array of medical service providers and places where healthcare occurs, including urgent care centers, rehabilitation centers, nursing homes, and long-term care facilities. In examples, outpatient care settings include settings where medical procedures, tests, and/or monitoring services are provided to patients without being admitted to a hospital, e.g., such as for an overnight hospital stay. Outpatient settings can include cardiology clinics, testing centers, providers of medical procedures on an outpatient basis, wellness and prevention services at outpatient clinics, rehabilitation centers, specialized outpatient service providers (e.g., hemodialysis, chemotherapy, etc.) or other similar care providers, and/or outpatient cardiac counseling program administrators or providers. Ambulatory patients in such clinical and/or outpatient settings can be prescribed a wearable defibrillator or a wearable cardioverter defibrillator (WCD). In-hospital care settings, on the other hand, include settings where medical procedures, tests, and/or monitoring services are provided to a patient on admission to a hospital, e.g., for an overnight hospital stay. Such in-hospital or inpatient care settings include emergency room (ER) visits and stays, intensive care unit (ICU) stays, or settings where patients are admitted to stay for a period of time (e.g., overnight), whether briefly or for an extended period of time. Patients in such in-hospital or inpatient settings can be prescribed a hospital wearable defibrillator (HWD), also described in further detail below.

A wearable cardiac treatment device, such as a WCD or an HWD, includes therapy electrodes or defibrillator pads positioned on an upper torso of a patient. In the case of a garment-based WCD, the therapy electrodes are disposed within a garment worn about the upper torso of the patient as described in further detail below. In the case of an adhesively-attached WCD or, for example, an HWD, the therapy electrodes are disposed within pads that are adhesively attached to the upper torso of the patient. The device is configured to continuously monitor the patient's heart to detect the heart rhythm. In the event a lethal cardiac arrhythmia is detected, the device can provide the patient with predetermined alarms, e.g., a vibration and/or gong alert that indicates the patient's attention is required and that a therapeutic shock is imminent. The patient can respond to the alarms by pressing buttons or otherwise providing a response to the device to cause the device to suspend the shock. If the patient does not respond to the alarms within a configurable period of time (e.g., typically about 45 seconds to about 75 seconds), the device is configured to deliver the therapeutic shock, e.g., a defibrillation shock. The device can be configured to deliver multiple shocks in this manner so long as underlying cardiac signals indicate an ongoing arrhythmia condition in the patient.

In such wearable cardiac treatment devices, a defibrillation shock may delivered through multiple vectors formed from pairs of therapy electrodes. For example, a wearable defibrillator may include a first and a second therapy electrode positioned on the front of the patient (e.g., an anterior side of the patient) and a third and a fourth therapy electrode positioned on the back of the patient (e.g., a posterior side of the patient). For example, the anterior location includes a sternum location (relative to the patient's heart). As another example, the anterior location includes location below the right clavicle and to the right of the sternum above the nipple. The wearable defibrillator may deliver a first pulse via the first and third therapy electrodes and a second pulse via the second and fourth therapy electrodes. As another example, a wearable defibrillator may include a first therapy electrode position on the front of the patient and a second and a third therapy electrode positioned on the back of the patient. The wearable defibrillator may deliver a first pulse via the first and second therapy electrodes and a second pulse via the first and third therapy electrodes.

As an illustration, a wearable defibrillator may include a first and a second therapy electrode positioned on the front of the patient (e.g., an anterior side of the patient) and a third and a fourth therapy electrode positioned on the lateral sides of the patient. In examples, the anterior location includes a sternum location (relative to the patient's heart). In examples, the anterior location includes location below the right clavicle and to the right of the sternum above the nipple. In examples, the lateral left side location includes a location on the lower left ribs in the midaxillary line. In examples, the lateral right side location includes a location on the lower right ribs in the midaxillary line. The wearable defibrillator may deliver a first pulse via the first and third therapy electrodes and a second pulse via the second and fourth therapy electrodes.

As another illustration, a wearable defibrillator may include a first and a second therapy electrode positioned on the front of the patient (e.g., an anterior side of the patient), a third and a fourth therapy electrode positioned on the back of the patient (e.g., a posterior side of the patient), and a fifth and a sixth therapy electrode positioned on the patient's left and right lateral sides. In examples, the lateral left side position includes an apex location (relative to the patient's heart). In examples, the lateral left side location includes a location on the lower left ribs in the midaxillary line. The wearable defibrillator may deliver a first pulse via the first and third therapy electrodes, a second pulse via the second and fourth therapy electrodes, and a third pulse via the first and fifth (or sixth) electrodes.

Additionally, in such wearable cardiac treatment devices, a defibrillation shock may be configured as one or more multiphasic therapeutic pulses, such as one or more biphasic therapeutic pulses. In a biphasic therapeutic pulse, a first portion of the pulse may be delivered with a positive polarity (e.g., a positive current from the perspective of a first therapy electrode to a second therapy electrode) and a second portion of the pulse may be delivered with a negative polarity (e.g., a negative current from the perspective of the first therapy electrode to the second therapy electrode). The two portions of the biphasic therapeutic pulse may be separated by a gap of predetermined length (e.g., a gap of around 0.01 ms to around 1 ms). The first portion and the second portion of the pulse may have the same or different shapes. These shapes may include a square or rectangular waveform, a sawtooth waveform, a truncated exponential waveform, and/or so on.

In examples, the defibrillation shock may be configured as one or more quadriphasic therapeutic shocks. In a quadriphasic therapeutic pulse, a first biphasic pulse may be delivered as described above, and further a second biphasic pulse may be delivered following the delivery of the first biphasic pulse. In examples, the portions of the quadriphasic therapeutic pulses may be separated by gaps that are each individually user-configurable or automatically configurable predetermined length (e.g., a gap of around 0.01 ms to around 100 ms). The first through fourth portions of the pulse may have the same or different shapes. As noted, these shapes may include a square or rectangular waveform, a sawtooth waveform, a truncated exponential waveform, and/or so on.

In examples, the defibrillation shock may be configured as multiple multiphasic therapeutic shocks that are delivered to the patient. In a multiphasic therapeutic pulse, two or more phases (e.g., with each phase having an opposite polarity from the previous phase) may be delivered to the patient, for example, as described above with reference to biphasic pulses. Two or more of these multiphasic therapeutic pulses may be delivered in succession, where the multiphasic therapeutic pulses are separated by predetermined delays (e.g., a delay of around 0.01 ms to around 100 ms between two multiphasic therapeutic pulses, such as between two biphasic therapeutic pulses). In examples, the predetermined delay may be set to 0 ms such that multiphasic therapeutic pulses may be delivered simultaneously via different vectors.

In examples, a wearable cardiac treatment device configured as a wearable defibrillator can be configured to deliver an electrical therapeutic pulse sequence to the patient though a first multiphasic therapeutic pulse (e.g., biphasic therapeutic pulse, triphasic therapeutic pulse, quadriphasic therapeutic pulse) delivered via a first vector formed by a first pair of therapy electrodes and a second multiphasic therapeutic pulse delivered via a second vector formed by a second pair of therapy electrodes. In such implementations, the wearable defibrillator may include at least one sensing electrode configured to monitor surface electric signals indicative of cardiac activity of the patient and therapeutic electrodes configured to deliver the therapeutic pulses to the patient. The wearable defibrillator may also include high-voltage circuitry connecting at least a first pair of the therapy electrodes and a second pair of the therapy electrodes (e.g., connecting the two electrodes of the first pair together and also connecting the two electrodes of the second pair together). Additionally, a garment may be configured to be worn about a torso of the patient and additionally configured to house the at least one sensing electrode, the therapy electrodes, and the high-voltage circuitry.

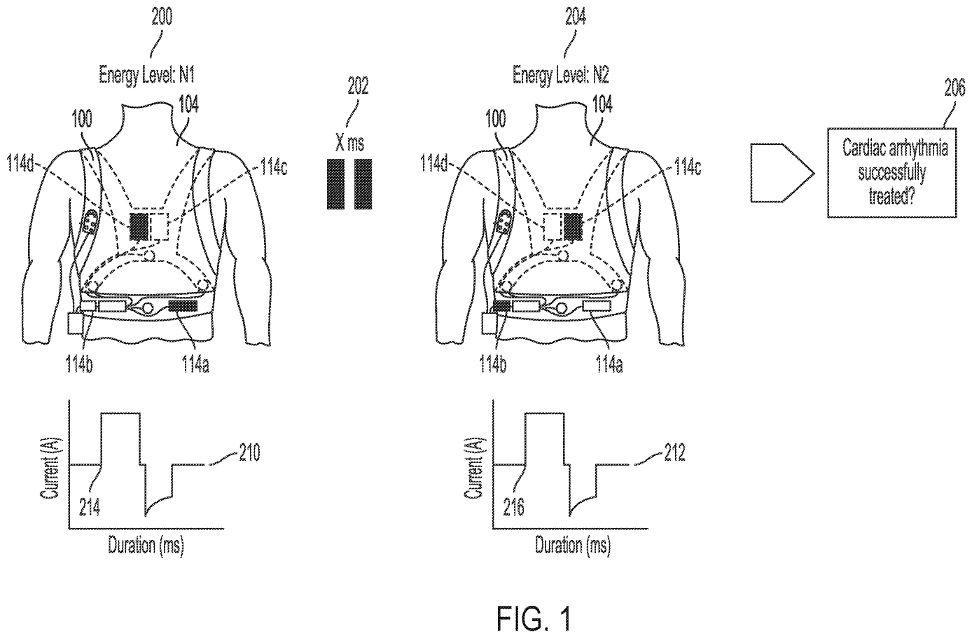

FIG. 1 illustrates an example of a treatment sequence for a patient 104 wearing such an ambulatory non-invasive wearable defibrillator 100 including therapy electrodes 114a-114d (e.g., non-adhesive therapy electrodes and/or adhesive therapy electrodes). Once the wearable defibrillator 100 has determined that the patient 104 is experiencing a treatable arrhythmia and should be delivered an electrical therapeutic treatment (e.g., after the patient has failed to respond to the vibratory, visual, and/or auditory alerts by pressing response buttons or otherwise indicating that shock should not be delivered), the wearable defibrillator 100 proceeds to a first portion 200 of an electrical therapeutic pulse sequence. In implementations, the wearable defibrillator 100 determines that the patient 104 should be delivered an electrical therapeutic treatment after outputting an alarm (e.g., tactile, visual and/or auditory) for the patient indicating that the wearable defibrillator 100 suspects that the patient is experiencing a cardiac arrhythmia condition and waiting for the expiry of a predetermined response period during which the patient fails to provide a response to the alert. During the first portion 200 of the electrical therapeutic sequence, the wearable defibrillator 100 delivers a first multiphasic therapeutic pulse at a first energy level via a first vector of the therapy electrodes 114a-114d. For example, as shown in FIG. 1, the wearable defibrillator 100 delivers a biphasic therapeutic pulse 210 at a first energy level (e.g., energy level N1) via a first vector of the therapy electrodes 114a-114d. For example, as shown in FIG. 1, the wearable defibrillator 100 may deliver the first biphasic therapeutic pulse 210 via a therapy electrode 114a positioned on the front side of the patient 104 and a therapy electrode 114d positioned on the back side of the patient 104. FIG. 1 also shows an example of the first biphasic therapeutic pulse 210 that the wearable defibrillator 100 may deliver to the patient 104. In this example, the first biphasic therapeutic pulse 210 includes a constant or mostly constant waveform in the first phase, followed by a gap, and further followed by a truncated exponential waveform in the second phase.

During a third portion 204 of the electrical therapeutic pulse sequence, the wearable defibrillator 100 delivers a second biphasic therapeutic pulse 212 at a second energy level via a second vector of the therapy electrodes 114a-114d. For example, as shown in FIG. 1, the wearable defibrillator 100 delivers a second biphasic therapeutic pulse 212 at a second energy level (e.g., energy level N2) via a second vector of the therapy electrodes 114a-114d. To illustrate, the wearable defibrillator 100 may deliver the second biphasic therapeutic pulse 212 via a therapy electrode 114b positioned on the front side of the patient 104 and a therapy electrode 114d positioned on the back side of the patient 104, as shown in FIG. 1. FIG. 1 also illustrates an example of the second biphasic therapeutic pulse 212, which in this example is configured similarly to the example of the first biphasic therapeutic pulse 210 with a constant or mostly constant waveform if a first phase, a gap, and a truncated exponential waveform in a second phase. In some implementations, however, the second biphasic therapeutic pulse 212 may have a different shape from the first biphasic therapeutic pulse 210.

Between the first portion 200 and the third portion 204 is a second portion 202 of the electrical therapeutic pulse sequence. The second portion 202 of the electrical therapeutic pulse sequence includes implementing a delay (e.g., X ms delay) between the delivery of first biphasic therapeutic pulse 210 and the second biphasic therapeutic pulse 212. The delay is determined between a leading edge 214 of the first biphasic therapeutic pulse 210 and a leading edge 216 of the second biphasic therapeutic pulse 212. In some implementations, the delay may be greater than or equal to the length of the first biphasic therapeutic pulse 210 such that the wearable defibrillator 100 delivers the first biphasic therapeutic pulse 210 followed by the second biphasic therapeutic pulse. In some implementations, the delay may be less than the length of the first biphasic therapeutic pulse 210 such that the wearable defibrillator 100 delivers the first biphasic therapeutic pulse 210 and the second biphasic therapeutic pulse 212 partially or completely simultaneously (e.g., there is complete or partial overlap between the first biphasic therapeutic pulse 210 and the second biphasic therapeutic pulse 212). Other examples and further details for the waveforms for the biphasic therapeutic pulses 210, 212, positions for therapeutic electrodes used to deliver the pulses, pairs of therapeutic electrodes used to deliver the pulses, pulse energy levels, and delays are discussed below.

After delivering the therapeutic pulses, the wearable defibrillator 100 determines whether the cardiac arrhythmia was successfully treated during a fourth portion 206 of the therapy delivery process. If the cardiac arrhythmia successfully treated the patient 104 (i.e., the cardiac rhythm of the patient 104 has returned to a normal sinus rhythm), the wearable defibrillator 100 may take no further action to treat the patient 104. However, if the wearable defibrillator 100 determines that the patient 104 is still experiencing a cardiac arrhythmia, the wearable defibrillator 100 may perform another electrical therapeutic pulse sequence (e.g., with higher energies being delivered at the first portion and/or the third portion of the sequence).

In one example use case, a clinician or other caregiver prescribes that a patient at risk of heart failure wear a wearable defibrillator for a certain amount of time (e.g., until the patient is scheduled for a surgery to receive an implantable cardiac defibrillator). If the wearable defibrillator determines that the patient is experiencing a suspected cardiac arrhythmia condition, the wearable defibrillator activates an alert for the patient. For example, the wearable defibrillator may activate a light alert, a sound alert (e.g., a siren, voice instructions telling the patient to press one or more response buttons, etc.), and/or a tactile alert. If the patient presses one or more response buttons within a predetermined response period, the wearable defibrillator delays or aborts a therapeutic treatment for the patient. If the patient does not press one or more response buttons within the predetermined response period, the wearable defibrillator initiates an electrical therapeutic pulse sequence. The electrical therapeutic pulse sequence includes delivering a first biphasic therapeutic pulse to the patient via a first vector formed by a first pair of the wearable defibrillator's therapy electrodes and delivering a second biphasic therapeutic pulse to the patient via second vector formed by a second pair of the wearable defibrillator's therapy electrodes, where the leading edges of the first and second biphasic therapeutic pulses are separated by a delay.

In implementations, the parameters of the electrical therapeutic pulse sequence may be preset to default values. In implementations, the prescribing clinician or other caregiver may be able to input values for the electrical therapeutic pulse sequence, such as the energy level for the first biphasic therapeutic pulse, the shape of the waveform for the first biphasic therapeutic pulse, the energy level for the second biphasic therapeutic pulse, the shape of the waveform for the second biphasic therapeutic pulse, the length of the delay, and/or the like. In implementations, the wearable defibrillator may automatically adjust at least some of the input values for the electrical therapeutic pulse sequence, such as based on impedance measurements taken from the patient.

The wearable defibrillators described herein may provide several advantages and benefits over prior art systems. For example, WCD or HWD systems can use biphasic shocks (e.g., 80 J to around 400 J) to convert a ventricular tachycardia (VT) or ventricular fibrillation (VF) event. In examples, a conversion success rate of the shocks can be used improve defibrillation efficacy by sequencing or overlapping the shocks via multiple vectors as described herein. Providing an electrical therapeutic treatment to the patient that includes sequential defibrillation of multiple multiphasic therapeutic pulses delivered through multiple therapy electrode vectors may be associated with better clinical outcomes for patients. For example, this multiphasic, multivector delivery (as described in implementation herein) may be more effective at terminating cardiac arrhythmias at lower energy values. Being able to successfully treat patients using lower-energy defibrillation may allow the wearable defibrillator to include less high-voltage circuitry, which may in turn allow the wearable defibrillator to have reduced size and weight. For example, capacitor sizes for the device may be designed to specifications that are smaller and lighter than for a single biphasic shock device. Such reduced weight and size wearable defibrillators may be lighter and more comfortable for the patient to wear (e.g., improve patient wearability and comfort). With improved wearability and comfort, patients may also be more likely to comply with wear prescriptions for the device (e.g., wearing the device for the recommended amount of time for each day of the prescribed use period). Additionally, these smaller and lighter high-voltage circuitry requirements may allow for improvements to the device manufacture and patient support infrastructure systems (e.g., in the process of supplying components and transporting them around the world up to the patient's location). As another example, delivering double sequential defibrillation via multiple vectors may be more effective at terminating a cardiac arrhythmia on the first delivery. Accordingly, patients may be more quickly treated and potentially suffer fewer side effects (e.g., burns from the electrical therapy) when the wearable defibrillator delivers the electrical therapeutic treatment.

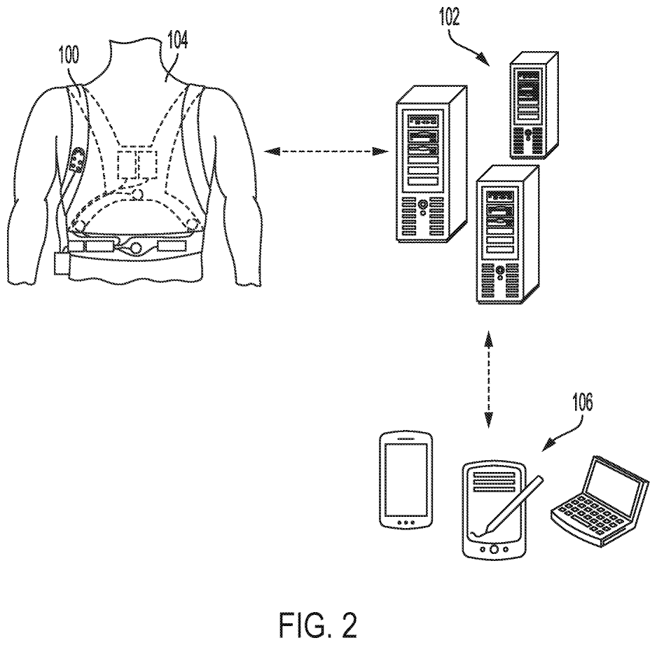

To further describe the wearable cardiac treatment system, FIG. 2 shows a system that includes the wearable defibrillator 100 configured to be worn by the patient 104. The wearable defibrillator 100 is in communication with a remote server 102. As discussed above, in implementations, the wearable defibrillator 100 may be implemented through a wearable garment configured to be worn about a torso of the patient 104. The wearable garment may be further configured to be worn continuously by the patient 104 for an extended period of time. Additionally, the wearable defibrillator 100 may include at least one sensing electrode, therapy electrodes, and high-voltage circuitry connecting pairs of the therapy electrodes, where the at least one sensing electrode, therapy electrodes, and the high-voltage circuitry are configured to be housed in the garment. In implementations, the wearable defibrillator 100 may include one or more other externally worn sensors configured to be disposed on the garment and output one or more physiological signals for the patient 104 and/or for the environment of the patient 104, such as vibrational sensors (e.g., biovibrational or cardiovibrational sensors configured to detect heart sounds), photoplethysmography sensors, radiofrequency (RF) sensors (which may be used, for example, to determine lung fluid content in the patient 104), temperature sensors, humidity sensors, and/or the like.

The wearable defibrillator 100 is configured to transmit signals and data generated by the wearable defibrillator 100 to the remote server 102. Accordingly, the wearable defibrillator 100 may be in wireless communication with the remote server 102. As an illustration, the wearable defibrillator 100 may communicate with the remote server 102 via cellular networks, via Bluetooth®-to-TCP/IP access point communication, via Wi-Fi, and the like. As such, the wearable defibrillator 100 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the wearable defibrillator 100 may be part of an Internet of Things (IoT) and communicate with the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

The remote server 102 is configured to receive and, in implementations, store and process the signals and data transmitted by the wearable defibrillator 100 worn by the ambulatory patient 104. Accordingly, the remote server 102 may include a computing device, or a network of computing devices, including at least one database (e.g., implemented in non-transitory computer-readable media or memory) and at least one processor configured to execute sequences of instructions (e.g., stored in the database, with the at least one processor being in communication with the database). The sequences of instructions may be configured to receive and process the signals transmitted by the wearable defibrillator 100. The at least one processor of the remote server 102 can be, for example, a digital signal processor (DSP) such as a 24-bit DSP processor. As another example, the at least one processor can be a multi-core processor, e.g., having two or more processing cores. As another example, the processor 916 can be an Advanced RISC Machine (ARM) processor, such as a 32-bit ARM processor. The at least one processor can execute an embedded operating system and further execute services provided by an operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others.

As further shown in FIG. 2, in implementations, the wearable cardiac treatment system may include one or more user interfaces, such as one or more clinician-authorized user terminals 106. The user terminals 106 are in electronic communication with the remote server 102 through a wired or wireless connection. For instance, the user terminals 106 may communicate with the remote server 102 via Wi-Fi, via Ethernet, via cellular networks, and/or the like. The user terminals 106 may include, for example, desktop computers, laptop computers, and/or portable personal digital assistants (e.g., smartphones, tablet computers, etc.).

The one or more clinician-authorized user terminals 106 are configured to electronically communicate with the remote server 102 for the purpose of sending and receiving information relating to the patient 104 wearing the wearable defibrillator 100. In implementations, the user terminals 106 are configured to allow clinicians to view information on the patient 104 wearing the wearable defibrillator 100. For example a user terminal 106 may display to the user (e.g., a clinician or other caregiver associated with the patient 104) information from a baselining therapy energy session conducted with the patient 104. In implementations, the user terminals 106 may display additional information about the wearable defibrillator 100 and/or the patient 104, such as one or more reports summarizing arrhythmia information for the patient 104, health information for the patient 104 (e.g., activity information for the patient 104, sleep information for the patient 104), wear status information for the patient 104 (e.g., how many hours per day the patient 104 wears the wearable defibrillator 100), and/or the like.

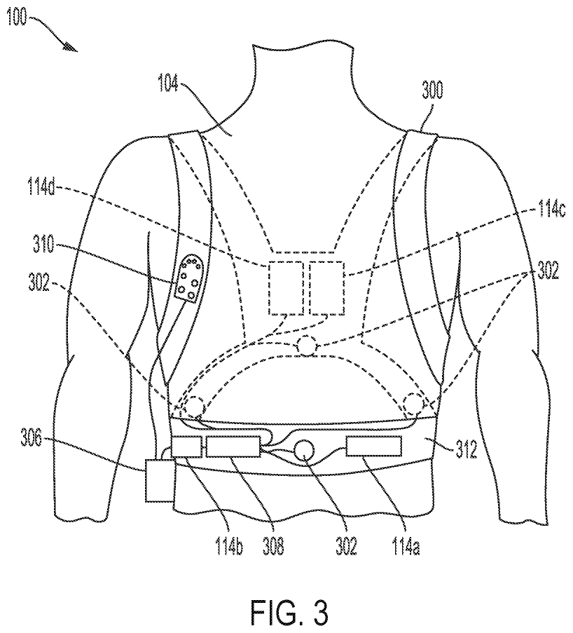

FIG. 3 shows the wearable defibrillator 100, according to implementations. As shown in FIG. 3, the wearable defibrillator 100 is external and wearable by the patient 104 around the patient's torso. Such a wearable defibrillator 100 can be, for example, capable and designed for moving with the patient 104 as the patient 104 goes about their daily routine. For example, the wearable defibrillator 100 may be configured to be bodily-attached to the patient 104. The wearable defibrillator 100 may be a wearable defibrillator or a wearable cardioverter defibrillator. In one example scenario, such wearable defibrillators can be worn nearly continuously or substantially continuously for a week, two weeks, a month, or two or three months at a time. During the period of time in which they are worn by the patient 104, the wearable defibrillators can be configured to continuously or substantially continuously monitor the vital signs of the patient 104 and can be configured to, upon determination that treatment is required, deliver one or more therapeutic electrical pulses to the patient 104. For example, such therapeutic shocks can be pacing, defibrillation, cardioversion, or transcutaneous electrical nerve stimulation (TENS) pulses.

As shown in FIG. 3, the wearable defibrillator 100 can include one or more of the following: a garment 300 configured to be worn about the patient's torso, at least one ECG sensing electrode 302 configured to be disposed on the garment 300 and further configured to monitor surface electric signals indicative of cardiac activity of the patient 104, one or more therapy electrodes 114a, 114b, 114c, and 114d (collectively referred to herein as therapy electrodes 114) configured to be disposed on the garment 300, a cardiac controller 306, a connection pod 308, a patient interface pod 310, a belt 312, or any combination of these. In implementations, the wearable defibrillator 100 may also include additional sensors, such as one or more motion detectors configured to generate motion data indicative of physical activity performed by the patient 104, one or more wear state sensors configured to detect a wear state of the wearable defibrillator 100, one or more vibrational or bioacoustics sensors configured to generate bioacoustics signals for the heart of the patient 104, one or more respiration sensors configured to generate respiration signals indicative of the respiration activity of the patient 104, and/or the like.

In examples, at least some of the components of the wearable defibrillator 100 can be configured to be disposed on the garment 300 by being removably mounted on or affixed to the garment 300, such as by mating hooks, hook-and-loop fabric strips, receptacles (e.g., pockets), snaps (e.g., plastic or metal snaps), and the like. For instance, the sensing electrodes 302 may be removably attached to the garment 300 by hook-and-loop fabric strips on the ECG sensing electrodes 302 and the garment 300, and the therapy electrodes 114 may be removably attached on the garment 300 by being inserted into receptacles of the garment 300. In some examples, at least some of the components of the wearable cardiac treatment device can be permanently integrated into the garment 300, such as by being sewn into the garment or by being adhesively secured to the garment 300 with a permanent adhesive. In examples, at least some of the components may be connected to each other through cables, through sewn-in connections (e.g., wires woven into the fabric of the garment 300), through conductive fabric of the garment 300, and/or the like.

The cardiac controller 306 can be operatively coupled to the sensing electrodes 302 and the therapy electrodes 114, which can be temporarily or removably affixed to the garment 300 (e.g., assembled into the garment 300 or removably attached to the garment 300, for example, using hook-and-loop fasteners) and/or permanently integrated into the garment 300 as discussed above. As shown in FIG. 3, the sensing electrodes 302 and/or the therapy electrodes 114 can be directly operatively coupled to the cardiac controller 306 and/or operatively coupled to the cardiac controller 306 through the connection pod 308. Component configurations other than those shown in FIG. 3 are also possible. For example, the ECG sensing electrodes 302 can be configured to be attached at various positions about the body of the patient 104. In some implementations, the ECG sensing electrodes 302 and/or at least one of the therapy electrodes 114 can be included on a single integrated patch and adhesively applied to the patient's body. In some implementations, the ECG sensing electrodes 302 and/or at least one of the therapy electrodes 114 can be included in multiple patches and adhesively applied to the patient's body. Such patches may be in a wired (e.g., via the connection pod 308) or wireless connection with the cardiac controller 306. Additionally, in implementations, the therapy electrodes 114 may be disposed into the garment 300 in different configurations, as discussed below in further detail.

As discussed above, the sensing electrodes 302 can be configured to monitor and detect surface electrical signals on the patient's skin that are indicative of cardiac activity of the patient 104 (i.e., ECG activity). Example ECG sensing electrodes 302 may include a metal electrode with an oxide coating such as tantalum pentoxide electrodes. For example, by design, the ECG sensing electrodes 302 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In implementations, the ECG sensing electrodes 302 can be used with an electrolytic gel dispersed between the electrode surface and the patient's skin. In implementations, the ECG sensing electrodes 302 can be dry electrodes that do not need an electrolytic material. As an example, such a dry electrode can be based on tantalum metal and having a tantalum pentoxide coating as is described above. Such dry electrodes can be more comfortable for long term monitoring applications.

In implementations, the ECG sensing electrodes 302 can include additional components such as accelerometers, acoustic signal detecting devices (e.g., vibrational sensors), and other measuring devices for recording additional parameters. For example, the ECG sensing electrodes 302 can also be configured to detect other types of patient physiological parameters and acoustic signals, such as tissue fluid levels, heart vibrations, lung vibrations, respiration vibrations, patient movement, etc. In implementations, the wearable defibrillator 100 may include sensors or detectors separate from the ECG sensing electrodes 302, such as separate motion detector(s), wear state detector(s), vibrational sensor(s), bioacoustics sensor(s), respiration sensor(s), temperature sensor(s), pressure sensor(s), and/or the like. In some examples, the therapy electrodes 114 can also be configured to include sensors configured to detect ECG signals as well as, or in the alternative, other physiological signals from the patient 104.

The connection pod 308 can, in some examples, include a signal processor configured to amplify, filter, and digitize cardiac signals, such as the ECG signals, prior to transmitting the cardiac signals to the cardiac controller 306. One or more therapy electrodes 114 can be configured to deliver one or more therapeutic cardioversion/defibrillation shocks to the body of the patient 104 when the wearable defibrillator 100 determines that such treatment is warranted based on the signals detected by the ECG sensing electrodes 302 and processed by the cardiac controller 306. Example therapy electrodes 114 can include conductive metal electrodes such as stainless-steel electrodes that include, in certain implementations, one or more conductive gel deployment devices configured to deliver conductive gel between the metal electrode and the patient's skin prior to delivery of a therapeutic shock.

In implementations, the cardiac controller 306 may also be configured to warn the patient 104 prior to the delivery of a therapeutic shock, such as via output devices integrated into or connected to the cardiac controller 306, the connection pod 308, and/or the patient interface pod 310. The warning may be auditory (e.g., a siren alarm, a voice instruction indicating that the patient 104 is going to be shocked), visual (e.g., flashing lights on the cardiac controller 306), haptic (e.g., a tactile, buzzing alarm generated by the connection pod 308), and/or the like. If the patient 104 is still conscious, the patient 104 may be able to delay or stop the delivery of the therapeutic shock. For example, the patient 104 may press one or more buttons on the patient interface pod 310 to indicate that the patient 104 is still conscious. In response to the patient 104 pushing the one or more buttons, the cardiac controller 306 may delay or stop the delivery of the therapeutic shock.

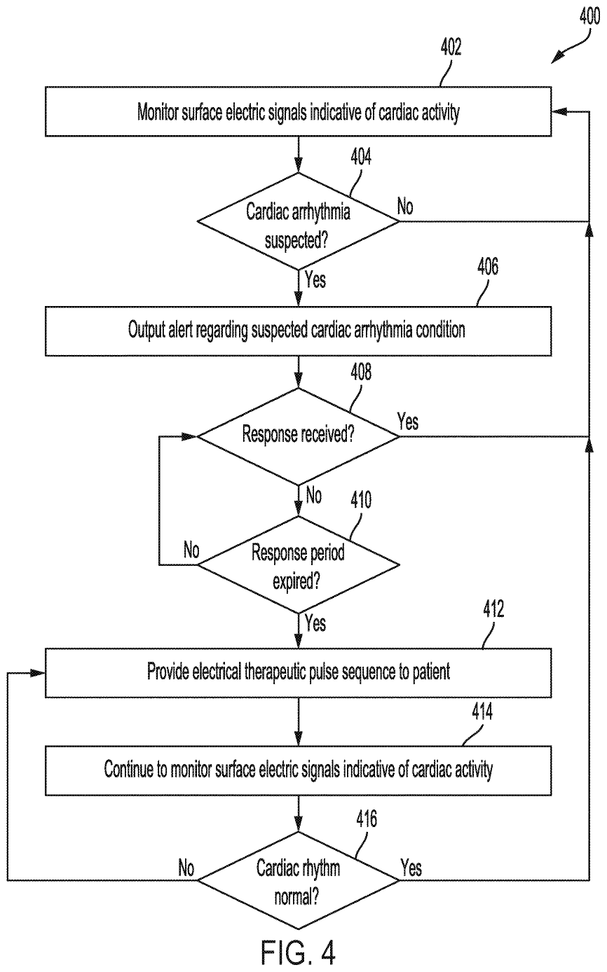

FIG. 4 illustrates a sample process flow for detecting and treating a suspected cardiac arrhythmia condition to a patient wearing a wearable defibrillator 100. The sample process 400 shown in FIG. 4 can be implemented by the cardiac controller 306, according to various implementations. As shown in FIG. 4, the cardiac controller 306 monitors surface electrical signals indicative of the cardiac activity of the patient 104 using the at least one sensing electrode 302 at step 402. The at least one sensing electrode 302 may detect the surface electrical signals from the skin of the patient 104. The cardiac controller 306 may receive sensed electrical signals from the at least one sensing electrode 302 and convert the sensed electrical signals into ECG signals, or the connection pod 308 may convert the sensed electrical signals into ECG signals, as discussed above.

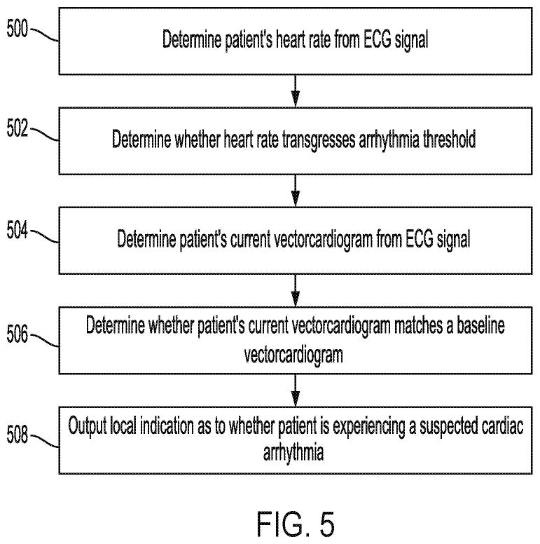

The cardiac controller 306 detects whether the patient 104 is experiencing a suspected cardiac arrhythmia condition based on the cardiac activity at step 404. FIG. 5 illustrates a sample process flow the cardiac controller 306 may use to perform step 404. As shown, the cardiac controller 306 determines the patient's heart rate from the ECG signal at step 500. The cardiac controller 306 may use a QRS detector on the ECG signal to determine the patient's heart rate. As another example, the cardiac controller 306 may determine the patient's heart rate by performing a fast Fourier transform (FFT) on the ECG signal or signals, with the FFT decomposing the analog ECG waveform into its frequency components. The cardiac controller 306 may then analyze the output of the FFT to determine the strongest frequency component indicative of heart rate. In implementations, the wearable defibrillator 100 may generate more than one ECG signal (e.g., at the cardiac controller 306, at the connection pod 308). For instance, the sensing electrodes 302 may form multiple ECG channels (e.g., form combinations of different pairs of the sensing electrodes 302), and the wearable defibrillator 100 may generate an ECG signal for each ECG channel. The cardiac controller 306 may use a QRS detector and analyze a fast Fourier transform (FFT) on each ECG signal to provide multiple independent assessments of the patient's heart rate.

In implementations, the cardiac controller 306 may use multiple methods to determine the patient's heart rate, for example, weighting the outputs of the methods to produce a final measure of the patient's heart rate. As an illustration, the cardiac controller 306 may apply logical weights based on comparing ECG channels, signal quality, and historic heart rate values to determine the best inputs for accurately monitoring the patient's heart rate. For example, if the heart rate from QRS detectors used on multiple ECG channels do not match, the cardiac controller 306 may apply less weight to these inputs and greater weight to other sources.

Once the cardiac controller 306 has determined the patient's heart rate, the cardiac controller 306 then determines if the patient's heart rate transgresses an arrhythmia threshold at step 502. For example, the cardiac controller 306 may determine if the patient's heart rate transgresses a threshold generally used for arrhythmias (e.g., 150, 160, 170, etc. bpm). As another example, the cardiac controller 306 may determine if the patient's heart rate transgresses a threshold used for a specific arrhythmia. To illustrate, the cardiac controller 306 may determine if the patient's heart rate is below a threshold for ventricular tachycardia, above the threshold for ventricular tachycardia but below a threshold for ventricular fibrillation, or above the threshold for ventricular fibrillation. As another illustration, the cardiac controller 306 may determine if the patient's heart rate is at or below a threshold for bradycardia, at or above a threshold for atrial fibrillation, at or above a threshold for ventricular tachycardia, and/or at or above a threshold for ventricular fibrillation. In implementations, these thresholds may be programmed for the patient 104 (e.g., by a technician or a clinician or other caregiver for the patient 104 during a setup period).

The cardiac controller 306 determines the patient's current vectorcardiogram from the ECG signal at step 504. For example, the sensing electrodes 302 may be positioned around the patient's torso when the patient 104 is wearing the wearable defibrillator 100 to form orthogonal leads (e.g., front-to-back and side-to-side at the level of the patient's xiphoid process). The cardiac controller 306 may determine a direction and magnitude of the electrical forces in the patient's heart and plot them (e.g., on an x-y or an x-y-z graph) to form a vectorcardiogram. In implementations, the cardiac controller 306 may determine the patient's vectorcardiogram if the patient's heart rate transgresses an arrhythmia threshold at step 502. In implementations, the cardiac controller 306 may determine the patient's current vectorcardiogram independent of whether the patient's heart rate transgresses an arrhythmia threshold at step 502. In implementations, the cardiac controller 306 may determine the patient's current vectorcardiogram as part of determining the patient's heart rate from the ECG signal. For instance, the cardiac controller 306 may plot the patient's current vectorcardiogram, determine the amount of time that it takes for the vectorcardiogram to repeat (e.g., for the plot to return to a starting point of within a certain vicinity of the starting point), and use that determination to output a heart rate for the patient 104.

The cardiac controller 306 determines whether the patient's current vectorcardiogram matches a baseline vectorcardiogram at step 506. To illustrate, the cardiac controller 306 may take a baseline vectorcardiogram for the patient 104 during a setup period and/or periodically during the patient's use of the wearable defibrillator 100 (e.g., weekly at a predetermined time, after the patient 104 is delivered a therapeutic shock, etc.). The cardiac controller 306 may then compare the patient's current vectorcardiogram to the patient's baseline vectorcardiogram to determine if the two morphologies match with a predetermined degree of accuracy. In implementations, the predetermined degree of accuracy may vary for the different types of arrhythmias that the cardiac controller 306 can detect. If the patient's current vectorcardiogram does not match their baseline vectorcardiogram, this failure to match may serve as evidence that the patient 104 is experiencing a suspected cardiac arrhythmia. If the patient's current vectorcardiogram does match their baseline vectorcardiogram with the predetermined degree of accuracy, this match may serve as evidence that the patient 104 is not experiencing a suspected cardiac arrhythmia. In implementations, the cardiac controller 306 may determine whether the patient's current vectorcardiogram matches their baseline vectorcardiogram only if the cardiac controller 306 has already determined that the patient's heart rate transgresses an arrhythmia threshold at step 502. In implementations, the cardiac controller 306 may determine whether the patient's current vectorcardiogram matches their baseline vectorcardiogram only if the cardiac controller 306 has already determined that the patient's heart rate transgresses an arrhythmia threshold at step 502. In implementations, the cardiac controller 306 may not use the patient's vectorcardiogram morphology, for example, if the signal quality from one of the sensing electrodes 302 is unreliable or if the patient's heart rate is above the ventricular fibrillation threshold. In such cases, the cardiac controller 306 may instead rely primarily on heart rate, stability (e.g., whether the R-R intervals of the patient's heart rate are consistent or inconsistent), onset criteria (e.g., whether the patient has experienced rapid changes in heart rate), and/or the like.

The cardiac controller 306 outputs an arrhythmia indication as to whether the patient 104 is experiencing a suspected cardiac arrhythmia at step 508. For example, if the patient's heart rate does not transgress an arrhythmia threshold, the cardiac controller 306 may output a local indication that the patient 104 is not experiencing a suspected cardiac arrhythmia. As another example, if the patient's heart rate transgresses an arrhythmia threshold but the patient's current vectorcardiogram matches their baseline vectorcardiogram with the predetermined degree of accuracy, the cardiac controller 306 may also output an arrhythmia indication that the patient 104 is not experiencing a suspected cardiac arrhythmia. As another example, if the patient's heart rate transgresses an arrhythmia threshold and the patient's current vectorcardiogram fails to match their baseline vectorcardiogram, the cardiac controller 306 may output an arrhythmia indication that the patient 104 is experiencing a suspected cardiac arrhythmia. As another example, if the patient's heart rate transgresses a first arrhythmia threshold (e.g., a threshold for ventricular fibrillation) but not a second arrhythmia threshold (e.g., a threshold for ventricular tachycardia that is higher than the threshold for ventricular fibrillation), and the patient's current vectorcardiogram fails to match their baseline vectorcardiogram, the cardiac controller 306 may output an arrhythmia indication that the patient 104 is experiencing a suspected cardiac arrhythmia. As another example, if the patient's heart rate transgresses an arrhythmia threshold corresponding to a non-treatable cardiac arrhythmia (e.g., a threshold for ventricular tachycardia), the cardiac controller 306 may output an arrhythmia indication that indication that the patient 104 is experiencing a non-treatable arrhythmia even if the patient's current vectorcardiogram fails to match their baseline vectorcardiogram.

In implementations, the cardiac controller 306 applies a confidence level as part of determining whether the patient is experiencing a suspected cardiac arrhythmia. As such, the cardiac controller 306 may assign weights to various inputs, such as the patient's heart rate, vectorcardiogram morphology, response button use (e.g., whether the patient 104 has already been alerted to a suspected cardiac arrhythmia and used a response button on the patient interface pod 310), signal quality, and/or the like to determine a confidence level for whether the patient is experiencing a suspected cardiac arrhythmia. The weighted inputs can contribute positively or negatively to the confidence level. If the cardiac controller 306 determines that an input is unreliable (e.g., signal quality is unreliable, different methods of determining the patient's heart rate produce different heart rate measures, etc.), the cardiac controller 306 may decrease the weight for that input. The cardiac controller 306 outputs an arrhythmia indication that the patient 104 is experiencing a suspected cardiac arrhythmia if the confidence level transgresses a predetermined confidence level threshold and otherwise outputs an arrhythmia indication that the patient is not experiencing a suspected cardiac arrhythmia.

Returning to FIG. 4, if the cardiac controller 306 determines that the patient 104 is not experiencing a suspected cardiac arrhythmia (e.g., outputs an arrhythmia indication that the patient 104 is not experiencing a suspected cardiac arrhythmia as a result of step 508 of FIG. 5), the cardiac controller 306 returns to monitoring the surface electric signals indicative of cardiac activity at step 402. If the cardiac controller 306 does determine that the patient 104 is experiencing a suspected cardiac arrhythmia (e.g., outputs an arrhythmia indication that the patient 104 is experiencing a suspected cardiac arrhythmia as a result of step 508 of FIG. 5), the cardiac controller 306 outputs an alert for the patient 104 regarding the suspected cardiac arrhythmia condition at step 406. For example, the cardiac controller 306 may activate an auditory alarm (e.g., output via a speaker of the cardiac controller 306). The auditory alarm may include a siren, instructions for the patient 104 to press one or more response buttons on the patient interface pod 310, and/or the like. As another example, the cardiac controller 306 may activate a visual alarm (e.g., a light integrated into the cardiac controller 306). As another example, the cardiac controller 306 may activate a tactile alarm (e.g., activate a vibration box of the connection pod 308). As another example, the cardiac controller 306 may activate a combination of an auditory alarm, a visual alarm, and/or a tactile alarm. In implementations, the cardiac controller 306 may also escalate the alert over time. For instance, the cardiac controller 306 may activate a siren, a light, and voice instructions for a certain amount of time and increase the volume of the siren after that amount of time has passed. As another illustration, the cardiac controller 306 may activate a siren and voice instructions for a certain amount of time and further activate a tactile alarm after that amount of time has passed. As another illustration, the cardiac controller 306 may activate a tactile alarm, after a first amount of time additionally activate a siren alarm, after a second amount of time increase the siren alarm, and after a third amount of time additionally activate an auditory prompt for the patient to press one or more response buttons to avoid treatment.

After the cardiac controller 306 activates the alert regarding the suspected cardiac arrhythmia condition, the cardiac controller 306 determines whether the wearable defibrillator 100 has received a response to the alert at step 408. If the cardiac controller 306 determines that the wearable defibrillator 100 has received a response to the alert (e.g., an input to one or more response buttons), the cardiac controller 306 returns to monitoring the surface electric signals indicative of cardiac activity at step 402. If the cardiac controller 306 determines that the wearable defibrillator 100 has not received a response to the alert, the cardiac controller 306 determines whether a predetermined response period has expired at step 410. For example, the predetermined response period be 10 to 100 s. In implementations, the predetermined response period may be configurable by a technician and/or a clinician or other caregiver.

If the cardiac controller 306 determines that the predetermined response period has not expired, the cardiac controller 306 continues to determine whether the wearable defibrillator 100 has received a response to the alert at step 408. If the cardiac controller 306 instead determines that the predetermined response period has expired with the patient 104 failing to provide a response to the alert, the cardiac controller 306 provides an electrical therapeutic pulse sequence to the patient 104 at step 412. For example, the predetermined response period may be around 20 to 40 seconds such that the cardiac controller 306 delivers the electrical therapeutic pulse sequence to the patient 104 within 20 to 40 seconds of detecting the suspected cardiac arrhythmia condition. As another example, the predetermined response period may be 10 to 20 seconds. As another example, the predetermined response period may be 5 to 10 seconds.

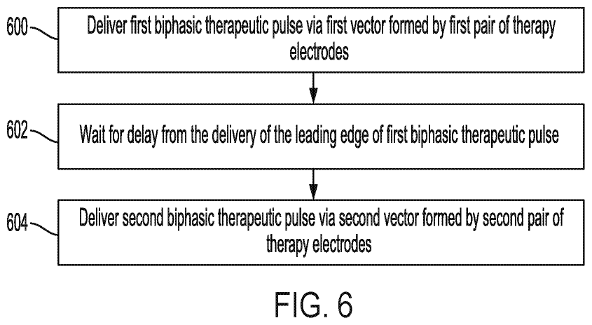

At step 412, the cardiac controller 306 delivers multiple multiphasic therapeutic pulses via multiple vectors formed of pairings of the therapy electrodes 114. FIG. 6 illustrates a sample process flow the cardiac controller 306 may use to perform step 412. As shown, as part of the electrical therapeutic pulse sequence, the cardiac controller 306 may deliver a first biphasic therapeutic pulse via a first vector formed by a first pair of the therapy electrodes 114 at step 600. The cardiac controller 306 waits for a delated from the delivery of the leading edge of the first biphasic therapeutic pulse at step 602. The cardiac controller 306 then delivers a second biphasic therapeutic pulse via a second vector formed by a second pair of the therapy electrodes 114 at step 604. As such, a timing of the electrical therapeutic pulse sequence as shown in FIG. 6 includes a first leading edge of the first biphasic therapeutic pulse being delivered at a first predetermined time and a second leading edge of the second biphasic therapeutic pulse being delivered at a second predetermined time following a delay after the first predetermined time.

In implementations, the first biphasic therapeutic pulse and the second biphasic therapeutic pulse may be defibrillation shocks. In implementations, the first biphasic therapeutic pulse and the second biphasic therapeutic pulse may be cardioversion shocks. For instance, the cardiac controller 306 may be configured to detect T-wave timings and/or R-waves timings of the patient's ECG signals (e.g., which the cardiac controller 306 uses to predict T-wave timings of the patient's ECG signals). The cardiac controller 306 may then use the T-wave timings and/or T-wave timings to avoid delivering the first biphasic therapeutic pulse and the second biphasic therapeutic pulse on the patient's T-waves. In implementations, the first biphasic therapeutic pulse and/or the second biphasic therapeutic pulse may be a pacing shock delivered to the patient 104. As an example, the first biphasic therapeutic pulse may be a pacing shock configured to establish a regular heart rhythm, and the second biphasic therapeutic pulse may be a defibrillation and/or cardioversion shock.

In implementations, the first biphasic therapeutic pulse may be delivered at an energy level of less than around 100 J (e.g., within a certain percentage or amount from 100 J, such as within 5%, 10%, 15%, 20% etc. and/or ±5 J, 10 J, 15 J, 20 J, etc. of 100 J), and the second biphasic therapeutic pulse may be delivered at an energy level of less than around 100 J. In implementations, the combined energy levels of the first biphasic therapeutic pulse and the second biphasic therapeutic pulse may be less than around 100 J. In implementations, the combined energy levels of the first biphasic therapeutic pulse and the second biphasic therapeutic pulse may be less than around 90 J. In implementations, the combined energy levels of the first biphasic therapeutic pulse and the second biphasic therapeutic pulse may be less than around 80 J. In implementations, the waveform of the first biphasic therapeutic pulse may last between around 10 ms and 50 ms, 10 m and 60 ms, 10 ms and 70 ms, 10 ms and 80 ms, 10 ms and 90 ms, 10 ms and 100 ms, and so on. In implementations, the waveform of the second biphasic therapeutic pulse may last between 10 ms and 50 ms, 10 m and 60 ms, 10 ms and 70 ms, 10 ms and 80 ms, 10 ms and 90 ms, 10 ms and 100 ms, and so on. The waveform of the second biphasic therapeutic pulse may be the same or nearly the same length as the waveform of the first biphasic therapeutic pulse, or the waveform of the second biphasic therapeutic pulse may be different (e.g., greater than or less than) the waveform of the first biphasic therapeutic pulse.

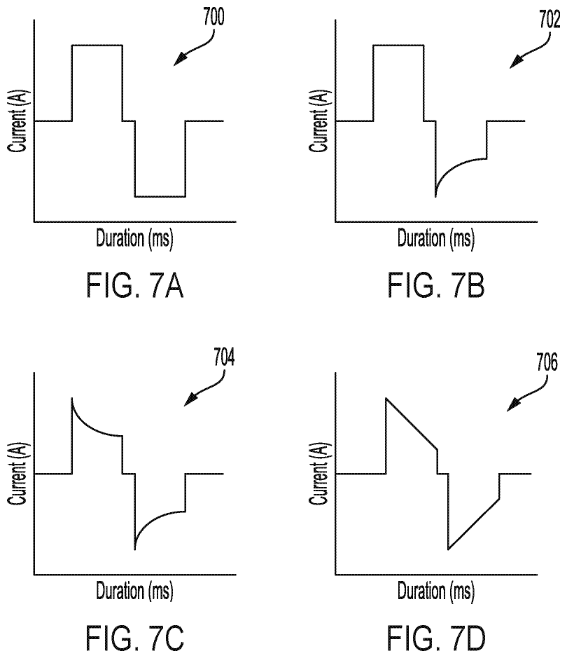

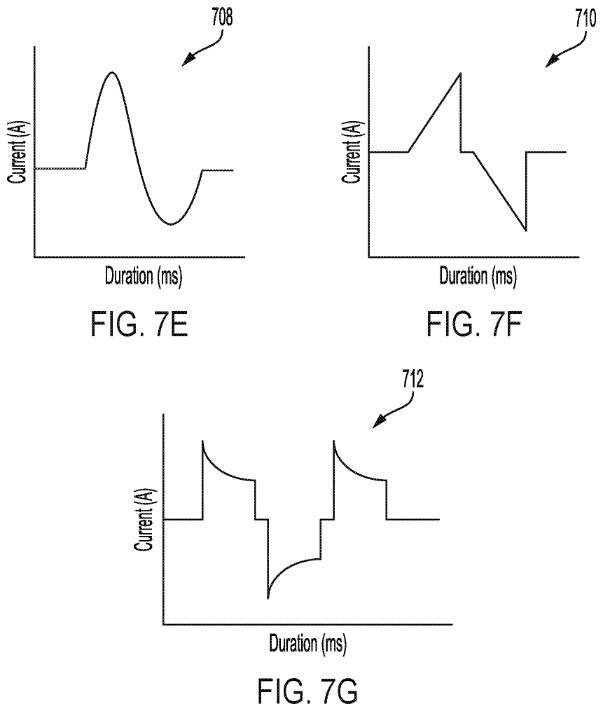

FIGS. 7A-7F illustrate examples of biphasic waveforms that may be delivered by the cardiac controller 306 via the therapy electrodes 114 as part of the first biphasic therapeutic pulse and/or second biphasic therapeutic pulse. FIG. 7A shows an example biphasic waveform 700 where the first phase is a constant current wave (e.g., a rectangular or square wave) and the second phase is a constant current wave of opposite polarity. FIG. 7B shows an example biphasic waveform 702 where the first phase is a constant current wave and the second phase is a truncated exponential wave. FIG. 7C shows an example biphasic waveform 704 where the first phase is a truncated exponential wave and the second phase is a truncated exponential wave of opposite polarity. FIG. 7D shows an example biphasic waveform 706 where the first phase is a sawtooth wave and the second phase is a sawtooth wave of opposite polarity. FIG. 7E shows an example biphasic waveform 708 where the phases are part of a dampened sinusoidal wave. FIG. 7F shows an example biphasic waveform 710 where the first phase is an ascending ramp and the second phase is an ascending ramp of opposite polarity.

Other biphasic waveforms may also be possible, such as waveforms where one or both phases of the waveform include a square wave, a rectangular wave, a truncated exponential wave, a sawtooth wave, a triangular wave, a sine wave, and/or the like. In implementations, the first biphasic therapeutic pulse and the second biphasic therapeutic pulse may be the same waveform. For example, the first biphasic therapeutic pulse and the second biphasic therapeutic pulse may be example waveform 700. In implementations, the first biphasic therapeutic pulse and the second biphasic therapeutic pulse may be different waveforms. For example, the first biphasic therapeutic pulse may be example waveform 700, and the second biphasic therapeutic pulse may be example waveform 702.

In implementations, as shown in FIGS. 7A-7F, the biphasic waveform may include a gap between the first phase and the second phase. This gap may be predetermined. In examples, the gap may be configurable, such as by a technician or a clinician inputting settings for the biphasic therapeutic pulse via the cardiac controller 306. In implementations, the biphasic waveform may not include a gap such that the second biphasic therapeutic pulse is delivered immediately after the first biphasic therapeutic pulse. In implementations, the first phase and the second phase of the biphasic therapeutic shock may be the same or similar in terms of amplitude and/or duration, as shown in FIGS. 7A-7D. In implementations, the first phase and the second phase of the biphasic therapeutic shock may be configured differently in terms of amplitude and/or duration. For example, the first phase may be of a higher amplitude and/or a longer duration than the second phase.

In implementations, the amplitude and/or duration of the biphasic waveform, including the amplitude and/or duration of each phase of the biphasic waveform, may depend on the energy level for the biphasic therapeutic shock, with a higher amplitude and longer duration creating a higher energy level for the biphasic therapeutic shock. As an illustration, in implementations, the first biphasic therapeutic pulse may be delivered at a higher energy level than the second biphasic therapeutic pulse. For example, the energy level of the first biphasic therapeutic pulse may be around 5 J greater, 10 J greater, 15 J greater, 20 J greater, etc. than the energy level for the second biphasic therapeutic pulse. Accordingly, the amplitude may be higher and/or the duration may be longer in the first biphasic therapeutic pulse compared to the second biphasic therapeutic pulse. In implementations, the energy level(s) for the first biphasic therapeutic pulse and/or the second biphasic therapeutic pulse may be based on default settings with default energy levels for the wearable defibrillator 100. In implementations, the energy level(s) of the first biphasic therapeutic pulse and/or the second biphasic therapeutic pulse may be user-configurable. For example, a technician or clinician or other caregiver for the patient 104 may input (e.g., via the cardiac controller 306) a desired energy level for the first biphasic therapeutic pulse and/or the second biphasic therapeutic pulse. The cardiac controller 306 may then set the energy level for the first biphasic therapeutic pulse and/or the second biphasic therapeutic pulse based on the energy level user input. To illustrate, the cardiac controller 306 may then automatically adjust the length, duration, and/or type of wave(s) for the biphasic therapeutic pulse(s) based on the energy level user input.

In implementations, the cardiac controller 306 may be configured to calculate the energy of the second biphasic therapeutic pulse level following the predetermined first time (e.g., the time at which the leading edge of the first biphasic therapeutic pulse is delivered). As an illustration, the cardiac controller 306 may monitor the patient's ECG during delivery of the first biphasic therapeutic pulse and modify the energy level of the second biphasic therapeutic pulse based on the patient's response to the first biphasic therapeutic pulse. For example, the cardiac controller 306 may identify whether the patient's ECG shows one or more indicators of a regular heart rhythm after deliver of the first biphasic therapeutic pulse and increase the energy level of the second biphasic therapeutic pulse if no indicators are identified and decrease the energy level of the second biphasic therapeutic pulse if at least one indicator is identified. As another illustration, the cardiac controller 306 may take an impedance measurement from the patient 104 during the delivery of the first biphasic therapeutic pulse and modify the energy level of the second biphasic therapeutic pulse to account for the measured impedance.

In implementations, the delay between the leading edge of the first biphasic therapeutic pulse and the second biphasic therapeutic pulse may be based on default settings with a default delay. In implementations, the delay may be user-configurable. For example, the cardiac controller 306 may receive a delay user input and set the delay based on the delay user input. In implementations, the delay may be greater than the duration or length of the first biphasic therapeutic pulse such that the second biphasic therapeutic pulse is delivered to the patient 104 after the conclusion of the first biphasic therapeutic pulse. In implementations, the delay may be less than or equal to the length of the first biphasic therapeutic pulse such that the second biphasic therapeutic pulse is delivered partially or wholly concurrently with the delivery of the first biphasic therapeutic pulse. As an example, the delay may be between around 0 ms and 250 ms. As another example, the delay may be between around 0 ms and 50 ms. As another example, the delay may be between around 10 ms and 250 ms. As another example, the delay may be between around 10 ms and 50 ms. As another example, the delay may be between around 120 ms and 150 ms.

Additionally, in implementations, the cardiac controller 306 may deliver a multiphasic therapeutic pulse having more than two phases, such as a triphasic or a quadriphasic therapeutic pulse. As such, FIG. 7G illustrates an example of a triphasic waveform 712 where the first phase is a truncated exponential wave, the second phase is a truncated exponential wave of opposite polarity, and the third phase is a truncated exponential wave having the first polarity. In examples, a quadriphasic therapeutic pulse could be formed from a similar waveform but include a fourth phase of a truncated exponential wave having the second, opposite polarity at the end.

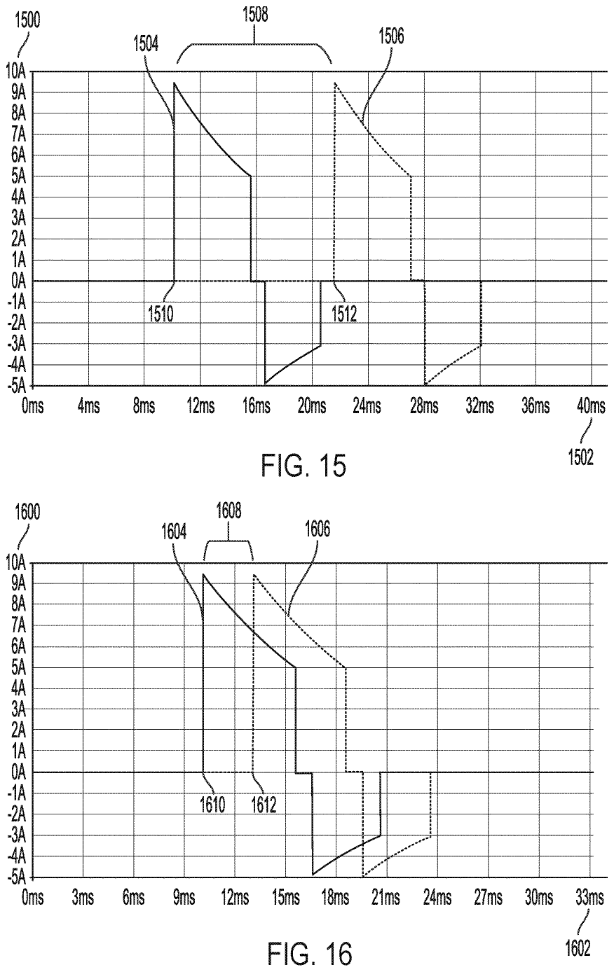

FIGS. 15 and 16 illustrate examples of multiple therapeutic waveforms of energy that may be delivered to a patient 104. FIG. 15 shows a chart of therapeutic amplitude 1500 versus waveform timing 1502. The chart includes a first therapeutic pulse 1504 and a second therapeutic pulse 1506, where each therapeutic pulse 1504, 1506 is configured as a biphasic waveform with each phase being a truncated exponential wave (e.g., similar to the waveform 704 shown in FIG. 7C). In addition, the amplitude of the first phase of the therapeutic pulses 1504, 1506 is greater than the amplitude of the second phase of the therapeutic pulses 1504, 1506. These configurations of the therapeutic pulses 1504 and 1506 are examples, however. In implementations, the wearable defibrillator 100 could deliver to the patient 104 a waveform with another configuration, such as one of the waveforms shown in FIGS. 7A-7B or 7D-7F. As shown in FIG. 15, there is no overlap between delivery of the therapeutic pulses 1504 and 1506; the second therapeutic pulse 1506 is delivered after the delivery of the first therapeutic pulse 1504 is completed. Accordingly, a delay 1508 between a leading edge 1510 of the first therapeutic pulse 1510 and a leading edge 1512 of the second therapeutic pulse 1506 is greater than the length of the first therapeutic pulse 1504.

FIG. 16 also shows a chart of therapeutic amplitude 1600 versus waveform timing 1602 with a first therapeutic pulse 1604 and a second therapeutic pulse 1606 configured similarly to the first therapeutic pulse 1504 and the second therapeutic pulse 1506 shown in FIG. 15. However, unlike the therapeutic pulses 1504 and 1506, the first therapeutic pulse 1604 and the second therapeutic pulse 1606 are delivered partially simultaneously. As such, a delay 1608 between a leading edge 1610 of the first therapeutic pulse 1604 and a leading edge 1612 of the second therapeutic pulse 1606 is less than the length of the first therapeutic pulse. In implementations, instead of being delivered partially simultaneously such that there is some overlap between the therapeutic pulses, the cardiac controller 306 may instead deliver the therapeutic pulses completely simultaneously.

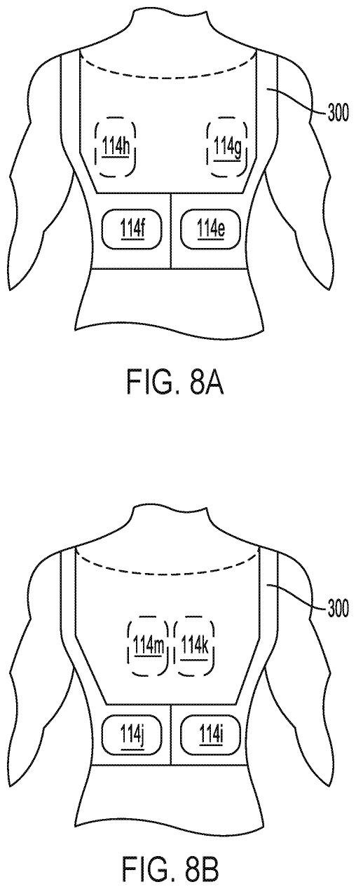

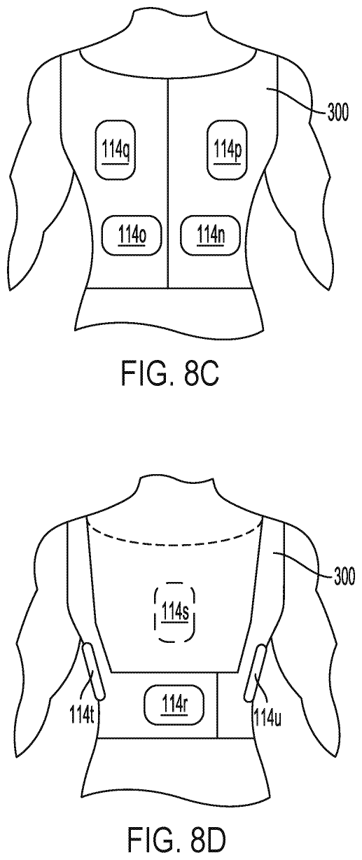

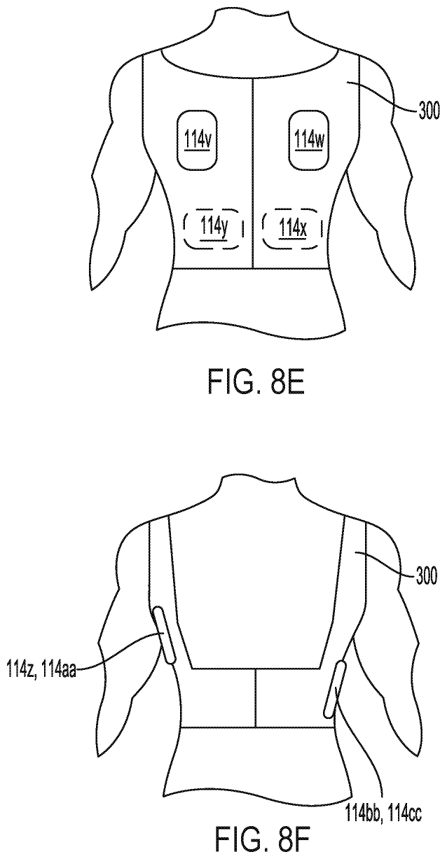

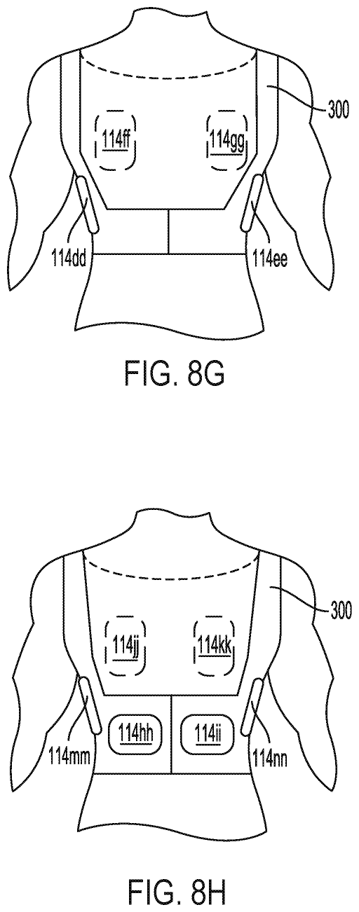

FIGS. 8A-8H illustrate example placements for the therapy electrodes 114 on the wearable defibrillator 100. FIG. 8A shows example placements where therapy electrodes 114e and 114f are configured to be positioned on an anterior portion of the patient's torso and where therapy electrodes 114g and 114h are configured to be positioned on a posterior portion of the patient's torso. Similarly, FIG. 8B shows example placements where therapy electrodes 114i and 114j are configured to be positioned on an anterior portion of the patient's torso and where therapy electrodes 114k and 114m are configured to be positioned on a posterior portion of the patient's torso (e.g., closer to the sagittal plane of the patient 104 and the position of the therapy electrodes 114g and 114h shown in FIG. 8A). In implementations with respect to FIGS. 8A and 8B, the cardiac controller 306 may deliver the first biphasic therapeutic pulse via a first therapy electrode 114 positioned against the anterior portion of the patient's torso and a second therapy electrode 114 positioned against the posterior portion of the patient's torso. For example, the cardiac controller 306 may deliver the first biphasic therapeutic pulse via the therapy electrodes 114e and 114h pair or the therapy electrodes 114f and 114g pair according to the placements shown in FIG. 8A or via the therapy electrodes 114i and 114m pair or the therapy electrodes 114j and 114k pair according to the placements shown in FIG. 8B. The cardiac controller 306 may then deliver the second biphasic therapeutic pulse via a third therapy electrode 114 positioned against the anterior portion of the patient's torso and a fourth therapy electrode 114 positioned against the posterior portion of the patient's torso. Continuing with the previous example, for instance, the cardiac controller 306 may deliver the second biphasic therapeutic pulse via the other of the therapy electrodes 114e and 114h pair or the 114f and 114g pair according to the placements shown in FIG. 8A or the therapy electrodes 114i and 114m pair or the therapy electrodes 114j and 114k pair according to the placements shown in FIG. 8B.

FIG. 8C shows example placements where therapy electrodes 114n, 114o, 114p, and 114q are all configured to be positioned on anterior portions of the patient's torso, with therapy electrodes 114p and 114q positioned superior to therapy electrodes 114n and 114o. In implementations, with respect to FIG. 8C, the cardiac controller 306 may deliver the first biphasic therapeutic pulse via a first therapy electrode 114 and a second therapy electrode 114 positioned superior to the first therapy electrode 114. For example, the cardiac controller 306 may deliver the first biphasic therapeutic pulse via the therapy electrodes 114n and 114q pair or the therapy electrodes 114o and 114p pair according to the placement shown in FIG. 8C. The cardiac controller 306 may then deliver the second biphasic therapeutic pulse via a third therapy electrode 114 and a fourth therapy electrode 114 positioned superior to the third therapy electrode 114. Continuing with the previous example, the cardiac controller 306 may deliver the second biphasic therapeutic pulse via the other of other of the therapy electrodes 114n and 114q pair or the therapy electrodes 114o and 114p pair.

FIG. 8D shows example placements where therapy electrode 114r is configured to be positioned on an anterior portion of the patient's torso, therapy electrode 114s is configured to be positioned on a posterior portion of the patient's torso, therapy electrode 114t is configured to be positioned on a right side portion of the patient's torso, and therapy electrode 114u is configured to be positioned on a left side portion of the patient's torso. For example, the therapy electrode 114t can be placed substantially about a middle axillary line on the right side of the patient. As another example, the therapy electrode 114u can be placed substantially about a middle axillary line on the left side of the patient. In implementations, with respect to FIG. 8D, the cardiac controller 306 may deliver the first biphasic therapeutic pulse via the therapy electrodes 114r and 114s on the patient's anterior and posterior portions or via the therapy electrodes 114t and 114u on the patient's right and left side portions. The cardiac controller 306 may then deliver the second biphasic therapeutic pulse via the other pairing of the therapy electrodes 114r and 114s or therapy electrodes 114t and 114u.

FIG. 8E shows example placements where therapy electrodes 114v and 114w are configured to be positioned on anterior portions of the patient's torso and therapy electrodes 114x and 114y are configured to be positioned on posterior portions of the patient's torso inferior to the therapy electrodes 114v and 114w. In implementations, with respect to FIG. 8E, the cardiac controller 306 may deliver a first biphasic therapy pulse via a pairing of one of the anterior therapy electrodes 114v and 114w and one of the posterior therapy electrodes 114x and 114y. The cardiac controller 306 may then deliver the second biphasic therapeutic pulse via another pairing of one of the anterior therapy electrodes 114v and 114w and one of the posterior therapy electrodes 114x and 114y.

FIG. 8F shows example placements where therapy electrodes 114z and 114aa are configured to be positioned on a right side portion of the patient's torso (e.g., where therapy electrodes 114z and 114aa are placed side-by-side on the patient's right side portion) and therapy electrodes 114bb and 114cc are configured to be positioned on a left side portion of the patient's torso inferior to the therapy electrodes 114z and 114aa (e.g., where therapy electrodes 114bb and 114cc are placed side-by-side on the patient's left side portion). For example, the therapy electrodes 114z and 114aa can be placed on either side about a middle axillary line on the right side of the patient. As another example, the therapy electrodes 114bb and 114cc can be placed on either side about a middle axillary line on the left side of the patient. In implementations, with respect to FIG. 8F, the cardiac controller 306 may deliver a first biphasic therapeutic pulse via a pairing of one of the right side therapy electrodes 114z and 114aa and one of the left side portion therapy electrodes 114bb and 114cc. The cardiac controller 306 may then deliver the second biphasic therapeutic pulse via another pairing of one of the right side therapy electrodes 114z and 114aa and one of the left side portion therapy electrodes 114bb and 114cc.

FIG. 8G shows example placements where therapy electrode 114dd is configured to be positioned on a right side portion of the patient's torso, therapy electrode 114ee is configured to be positioned on a left side portion of the patient's torso, and therapy electrodes 114ff and 114gg are configured to be positioned on a posterior portion of the patient's torso. In implementations, with respect to FIG. 8G, the cardiac controller 306 may deliver a first biphasic therapeutic pulse via a first therapy electrode 114 positioned against the patient's side and a second therapy electrode 114 positioned against the posterior portion of the patient's torso. For example, the cardiac controller 306 may deliver the first biphasic therapeutic pulse via the therapy electrodes 114ee and 114ff or via the therapy electrodes 114dd and 114gg. For example, the therapy electrode 114dd can be placed substantially about a middle axillary line on the right side of the patient. As another example, the therapy electrode 114ee can be placed substantially about a middle axillary line on the left side of the patient. The cardiac controller 306 may then deliver the second biphasic therapeutic pulse via a third therapy electrode 114 positioned against the patient's side and a fourth therapy electrode 114 positioned against the posterior portion of the patient's torso. Continuing with the previous example, the cardiac controller 306 may deliver the second biphasic therapeutic pulse via the other of the therapy electrodes 114ee and 114ff pair or the therapy electrodes 114dd and 114gg pair.

FIG. 8H shows example placements where therapy electrodes 114hh and 114ii are configured to be positioned on an anterior portion of the patient's torso, therapy electrodes 114jj and 114kk are configured to be positioned against a posterior portion of the patient's torso, therapy electrode 114mm is configured to be positioned again a right side portion of the patient's torso, and therapy electrode 114nn is configured to be positioned against a left side portion of the patient's torso. For example, the therapy electrode 114mm can be placed substantially about a middle axillary line on the right side of the patient. As another example, the therapy electrode 114nn can be placed substantially about a middle axillary line on the left side of the patient. In implementations, with respect to FIG. 8H, the cardiac controller 306 may deliver the first biphasic therapeutic pulse via one pair of the electrodes 114hh-114nn and deliver the second biphasic therapeutic pulse via a second pair of the electrodes 114hh-114nn. For example, the pairs of therapy electrodes 114 may be 114hh and 114kk, 114ii and 114jj, and 114mm and 114nn.

In implementations, the garment 300 is configured to receive the therapy electrodes 114 in the positions shown in FIGS. 8A-8H. As an illustration, the garment 300 may include pockets configured to receive the therapy electrodes 114 as shown in FIGS. 8A-8H. Thus, for the positions shown in FIGS. 8A and 8B, the garment 300 may include two pockets configured to be positioned against an anterior portion of the patient's torso and configured to receive therapy electrodes 114e and 114f in FIG. 8A, 114i and 114j in FIG. 8B, or 114v and 114w in FIG. 8E. The garment 300 may also include two pockets configured to be positioned against a posterior portion of the patient's torso and configured to receive electrodes 114g and 114h in FIG. 8A, 114k and 114m in FIG. 8B, or 114x and 114y in FIG. 8E. For the positions shown in FIG. 8C, the garment 300 may include two pockets configured to be positioned against an anterior portion of the patient's torso and configured to receive therapy electrodes 114n and 114o. The garment 300 may additionally include two pockets configured to be positioned against an anterior portion of the patient's torso superior to the therapy electrodes 114n and 114o and configured to receive therapy electrodes 114p and 114q. For the positions shown in FIG. 8D, the garment 300 may include a pocket configured to be positioned against an anterior portion of the patient's torso and configured to receive therapy electrode 114r, a pocket configured to be positioned against a posterior portion of the patient's torso and configured to receive therapy electrode 114s, a pocket configured to be positioned against a right side portion of the patient's torso and configured to receive therapy electrode 114t, and a pocket configured to be positioned against a left side portion of the patient's torso and configured to receive therapy electrode 114u. For the positions shown in FIG. 8F, the garment 300 may include two pockets configured to be positioned against a right side portion of the patient's torso and configured to receive therapy electrodes 114z and 114aa and two pockets configured to be positioned against a left side portion of the patient's torso and configured to receive therapy electrodes 114bb and 114cc.

In implementations, the electrode placements may be selected to account for male and female anatomy. For example, the electrode placements shown in FIGS. 8C and 8E including therapy electrodes 114 placed against an upper anterior portion of the patient's torso may be used for male patients. The placement of the upper anterior therapy electrodes 114 may be adjusted for female patients to avoid interference from breast tissue. As an illustration, the upper anterior therapy electrodes 114 may be raised or moved to the patient's sides to avoid being placed on top of the patient's breast tissue.

Other positions of the therapy electrodes 114 may alternatively be used. For example, in implementations, the garment 300 may be configured to receive fewer than four or more than four therapy electrodes 114, such as the six therapy electrodes 114 configuration shown in FIG. 8H. As another example, in implementations, more than two therapy electrodes 114 may be configured to be positioned against a posterior portion of the patient's torso. As another example, in implementations, the wearable defibrillator 100 may include additional pairs of electrodes such that shock vectors that rotate around the patient's torso may be used. In implementations, a pair of therapy electrodes 114 may include two electrically coupled electrodes functioning as a single electrode, where the two electrically coupled electrodes are paired with another therapy electrode 114 to form the pair of therapy electrodes 114. For example, therapy electrodes 114k and 114m of FIG. 8B may be electrically coupled together to function as a single electrode. As such, the two vectors used for the electrical therapeutic pulse sequence may be therapy electrode 114j and therapy electrodes 114k and 114m as a first pair and therapy electrode 114i and therapy electrodes 114k and 114m as a second pair. Alternatively, any of the single electrodes shown in FIGS. 8A-8H may be configured as two electrically coupled electrodes functioning as a single electrode.

In implementations, the positioning of the therapy electrodes 114 and the therapy electrode vectors used by the cardiac controller 306 to deliver the electrical therapeutic pulse sequence may be configured such that the first vector and the second vector of the electrical therapeutic pulse sequence are configured to be at a substantially orthogonal angle from each other. To illustrate, the first vector formed by the therapy electrodes 114 may extend from a first geometrical center of a first one of a first therapy electrode pair to a second geometrical center of a second one of the first therapy electrode pair. The second vector formed by the therapy electrodes 114 may extend from a third geometrical center of a first one of a second therapy electrode pair to a fourth geometrical center of a second one of the second therapy electrode pair. Projections of the first and second vectors onto a plane of the patient 104 may thus be substantially orthogonal to each other. For example, projections of the first and second vectors onto a transverse plane of the patient 104 may be substantially orthogonal to each other. As another example, projections of the first and second vectors onto a coronal plane of the patient 104 may be substantially orthogonal to each other. As another example, projections of the first and second vectors onto a sagittal plane of the patient 104 may be substantially orthogonal to each other. In implementations, the projections of the first and second vectors onto a plane of the patient 104 may be at an angle 50 to 150 degrees to each other. In implementations, the projections of the first and second vectors onto a plane of the patient 104 may be at an angle of 70 to 110 degrees to each other. In implementations, the projections of the first and second vectors onto a plane of the patient 104 may be at an angle of 45 to 135 degrees to each other.

In implementations, the cardiac controller 306 may adjust at least one parameter of the electrical therapeutic pulse sequence based on a type of suspected cardiac arrhythmia condition the cardiac controller 306 determines that the patient 104 is experiencing. For example, the cardiac controller 306 may determine that the patient 104 is suspected of experiencing ventricular fibrillation or ventricular tachycardia at step 404 of FIG. 4. Based on whether the patient 104 is suspected of experiencing ventricular fibrillation or ventricular tachycardia, the cardiac controller 306 may adjust the first predetermined time at which the leading edge of the first biphasic therapeutic pulse is delivered, the delay, the energy level of the first biphasic therapeutic pulse, and/or the energy level of the second biphasic therapeutic pulse. For instance, if the cardiac controller 306 suspects that the patient 104 is experiencing ventricular tachycardia, the cardiac controller 306 may deliver the first biphasic therapeutic pulse as a pacing pulse and the second biphasic therapeutic pulse as a defibrillation or cardioversion pulse. As another example, if the cardiac controller 306 suspects that the patient 104 is experiencing ventricular tachycardia, the cardiac controller 306 may deliver cardioversion therapy, whereas the cardiac controller 306 may deliver asynchronous electrical therapy if the cardiac controller 306 suspects that the patient 104 is experiencing ventricular fibrillation.

In implementations, the electrical therapeutic pulses sequence may include more than two therapeutic pulses (e.g., a third biphasic therapeutic pulse, a third and a fourth biphasic therapeutic pulse, etc.). For example, the electrical therapeutic pulse sequence may include a third biphasic therapeutic pulse delivered at a third energy level, where a leading edge of the third biphasic therapeutic pulse is delivered at a third predetermined time and separated from the leading edge of the second biphasic therapeutic pulses by a second predetermined delay. The second predetermined delay may be the same as the first predetermined delay, or the second predetermined delay may be different from the first predetermined delay. The cardiac controller 306 may deliver the third biphasic therapeutic pulse using the same vectors formed by the therapy electrodes 114 (e.g., the same vectors used for the first and/or second biphasic therapeutic pulse), or the cardiac controller 306 may deliver the third biphasic therapeutic pulse using at least one vector different from the first and second biphasic therapeutic pulses. For example, the wearable defibrillator 100 may include more than four therapy electrodes 114 such that additional vectors may be used.

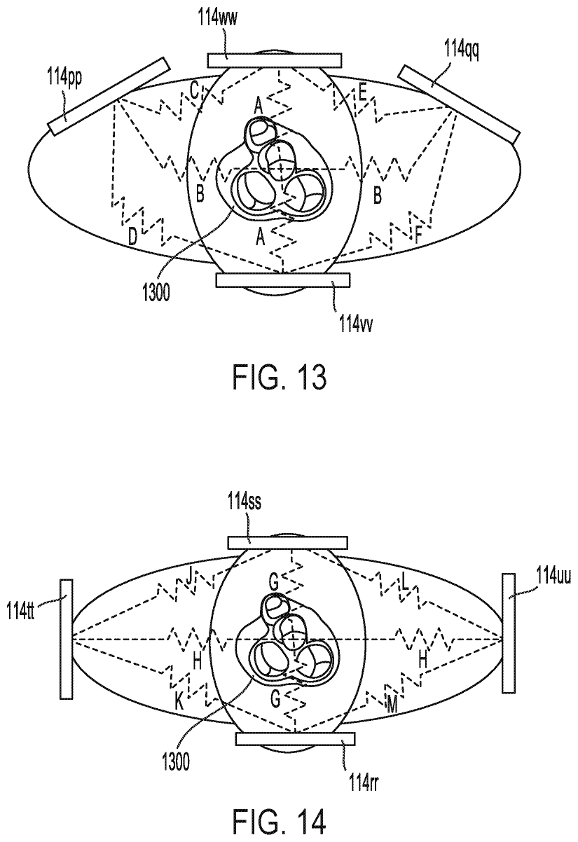

FIGS. 13 and 14 illustrate examples of vectors that may be created by placement of multiple therapy electrodes 114 (e.g., in a similar configuration as shown in FIG. 8D). FIG. 13 shows example vectors that may be created with placement of a therapy electrode 114vv on an anterior portion of the patient's torso, a therapy electrode 114ww on a posterior portion of the patient's torso, a therapy electrode 114pp on a right posterior portion of the patient's torso, and a therapy electrode 114qq on a left posterior portion of the patient's torso. Accordingly, multiple vectors may be formed between the therapy electrodes 114vv, 114ww, 114pp, and 114qq that the wearable defibrillator 100 may use to deliver therapeutic shocks to the patient's heart 1300. As shown in FIG. 13, these vectors may include (1) an A vector formed between the anterior therapy electrode 114mm and posterior therapy electrode 114ww, (2) a B vector formed between the right posterior therapy electrode 114pp and left posterior therapy electrode 114qq, (3) a C vector formed between the posterior therapy electrode 114ww and right posterior therapy electrode 114pp, (4) a D vector formed between the anterior therapy electrode 114vv and the right posterior therapy electrode 114pp, (5) an E vector formed between the posterior therapy electrode 114ww and the left posterior therapy electrode 114qq, and (6) an F vector formed between the anterior therapy electrode 114vv and the left posterior therapy electrode 114qq. In delivering multiple sequential defibrillation therapy, the cardiac controller 306 may activate delivery of therapeutic shocks via some or all of these vectors A-F.

Similarly, FIG. 14 shows example vectors that may be created with placement of a therapy electrode 114rr on an anterior portion of the patient's torso, a therapy electrode 114ss on a posterior portion of the patient's torso, a therapy electrode 114tt on a right portion of the patient's torso, and a therapy electrode 114uu on a left portion of the patient's torso. As with FIG. 13, multiple vectors may be formed between the therapy electrodes 114rr, 114ss, 114tt, and 114uu that the wearable defibrillator 100 may use to deliver therapeutic shocks to the patient's heart 1300. These vectors may include (1) a G vector formed between the anterior therapy electrode 114rr and the posterior therapy electrode 114ss, (2) an H vector formed between the right therapy electrode 114tt and the left therapy electrode 114uu, (3) a J vector formed between the posterior therapy electrode 114ss and the right therapy electrode 114tt, (4) a K vector formed between the anterior therapy electrode 114rr and the right therapy electrode 114tt, (5) an L vector formed between the posterior therapy electrode 114ss and the left therapy electrode 114uu, and (6) an M vector formed between the anterior therapy electrode 114rr and the left therapy electrode 114uu. As with the system shown in FIG. 13, in delivering multiple sequential defibrillation therapy, the cardiac controller 306 may activate deliver of therapeutic shocks via some or all of these vectors G-M.

Returning to FIG. 4, after and/or during the delivery of the electrical therapeutic pulse sequence, the cardiac controller 306 is configured to continue monitoring the surface electric signals indicative of the cardiac activity of the patient 104 at step 414 (e.g., similar to the process discussed above with respect to step 402). The cardiac controller 306 is configured to determine, based on the continued monitoring, whether the patient's cardiac rhythm has returned to normal after the provided electrical therapeutic pulse sequence at step 416. The cardiac controller 306 may implement step 416 similarly to the process described above for step 404 (e.g., to determine if the patient 104 shows a continued suspected cardiac arrhythmia or if the patient's cardiac rhythm appears to be normal). If the cardiac controller 306 determines that the patient's cardiac rhythm has returned to normal, the cardiac controller 306 is configured to return to monitoring the patient's surface electric signals indicative of cardiac activity at step 402. If the cardiac controller 306 determines that the patient's cardiac rhythm has not returned to normal (e.g., that the patient 104 still has a suspected cardiac arrhythmia), the cardiac controller 306 is configured to provide another electrical therapeutic pulse sequence to the patient 104.

In implementations, the cardiac controller 306 may provide the second electrical therapeutic pulse sequence to the patient 104 using a similar process as described above with respect to FIG. 6. For example, the cardiac controller 306 may deliver a third biphasic therapeutic pulse and a fourth biphasic therapeutic pulse, where the leading edge of the third biphasic therapeutic pulse is delivered at a third predetermined time and the leading edge of the fourth biphasic therapeutic pulse is delivered at a fourth predetermined time following a second delay after the third predetermined time. In implementations, the cardiac controller 306 may provide the second electrical therapeutic pulse sequence using the same or similar waveform(s), delay, and therapy electrode vectors used for the first electrical therapeutic pulse sequence. In implementations, the cardiac controller 306 may use one or more different waveforms, delay, and/or one or more different therapy electrode vectors from the first electrical therapeutic pulse sequence. In implementations, combined energy level of the first electrical therapeutic pulse sequence may be different from the combined energy level of the second electrical therapeutic pulse sequence.

To illustrate, the cardiac controller 306 may use the same basic waveform(s) and therapy electrode vectors but adjust the amplitude and/or durations of the waveforms to provide higher energy levels in the third and/or fourth biphasic therapeutic pulses compared to the first and/or second biphasic therapeutic pulses. As an example, the cardiac controller 306 may configure the third biphasic therapeutic pulse such that the energy level of the third biphasic therapeutic pulse is higher than the energy level of the first biphasic therapeutic pulse and/or configure the fourth biphasic therapeutic pulse such that the energy level of the fourth biphasic therapeutic pulse is higher than the energy level of the second biphasic therapeutic pulse. For instance, the combined energy of the first electrical therapeutic pulse sequence may be less than around 80 J (e.g., between 60 to 80 J, between 60 to 70 J, etc.) and the combined energy of the second electrical therapeutic pulse sequence may be less than around 100 J (e.g., between 80 to 100 J, between 80 to 90 J, etc.)

As another illustration, the cardiac controller 306 may deliver the third biphasic therapeutic pulse via the first vector formed by the first pair of therapy electrodes 114 or via the second vector formed by the second pair of therapy electrodes 114 (e.g., according to the vectors described above with respect to steps 600 and 604 of FIG. 6). The cardiac controller 306 may then deliver the fourth biphasic therapeutic pulse via the other of the first vector or the second vector. As another illustration, the cardiac controller 306 may deliver the third biphasic therapeutic pulse via a third vector formed by a third pair of the therapy electrodes 114 and deliver the fourth biphasic therapeutic pulse via a fourth vector formed by a fourth pair of the therapy electrodes 114. As another illustration, the cardiac controller 306 may use a different delay in the second electrical therapeutic pulse sequence from the delay in the first electrical therapeutic pulse sequence (e.g., a smaller delay). As another illustration, the energy level delivery distribution of the first electrical therapeutic pulse sequence may be different from the energy level delivery distribution of the second electrical therapeutic pulse sequence. For example, the proportion of the energy level delivered via the first biphasic therapeutic pulse compared to the second biphasic therapeutic pulse may be different from the proportion of the energy level delivered via the third biphasic therapeutic pulse compared to the fourth biphasic therapeutic pulse.

As shown in FIG. 4, in implementations, the cardiac controller 306 continues to monitor the patient's electric signals indicative of cardiac activity and provide electrical therapeutic pulse sequences to the patient 104 until the cardiac controller 306 determines that the patient's cardiac rhythm has returned to normal. For instance, the cardiac controller 306 may continue providing electrical therapeutic pulse sequences with increasing combined therapeutic energy until the patient's cardiac rhythm returns to normal. In implementations, the cardiac controller 306 may adjust energy levels for a future electrical therapeutic pulse sequence once the cardiac controller 306 has determined that the patient's cardiac rhythm has returned to normal. As an illustration, if the cardiac controller 306 delivers two electrical therapeutic pulse sequences to the patient 104 before the patient's cardiac rhythm returns to normal, the cardiac controller 306 may adjust energy levels for any future electrical therapeutic pulse sequences to be delivered to the patient 104 based on the energy levels of the second electrical therapeutic pulse sequence delivered to the patient 104 (e.g., based on the energy levels of the third biphasic therapeutic pulse and the fourth biphasic therapeutic pulse). For example, the cardiac controller 306 may set a future electrical therapeutic pulse sequence to begin at the second electrical therapeutic pulse sequence.

In implementations, the cardiac controller 306 is configured to determine at least one impedance measurement for the patient 104 based on a provided electrical therapeutic pulse sequence. For instance, the cardiac controller 306 may measure the voltage between the pairs of electrodes forming the vectors used in an electrical therapeutic pulse sequence. Using the measured voltage, the current delivered via the vectors, and Ohm's Law, the cardiac controller 306 may determine an impedance for each of the vectors used during the electrical therapeutic pulse sequence. The cardiac controller 306 may then adjust at least one parameter for a future electrical therapeutic pulse sequence based on the at least one impedance measurement. For example, the cardiac controller 306 may adjust the current and/or duration of one or both of the biphasic therapeutic pulses for a future electrical therapeutic pulse sequence. In implementations, the cardiac controller 306 is configured to determine at least one intermediate impedance measurement for the patient 104 based on the first biphasic therapeutic pulse of an electrical therapeutic pulse sequence. The cardiac controller 306 may then similarly adjust at least one parameter of the second biphasic therapeutic pulse of the electrical therapeutic pulse sequence based on the at least one intermediate impedance measurement.

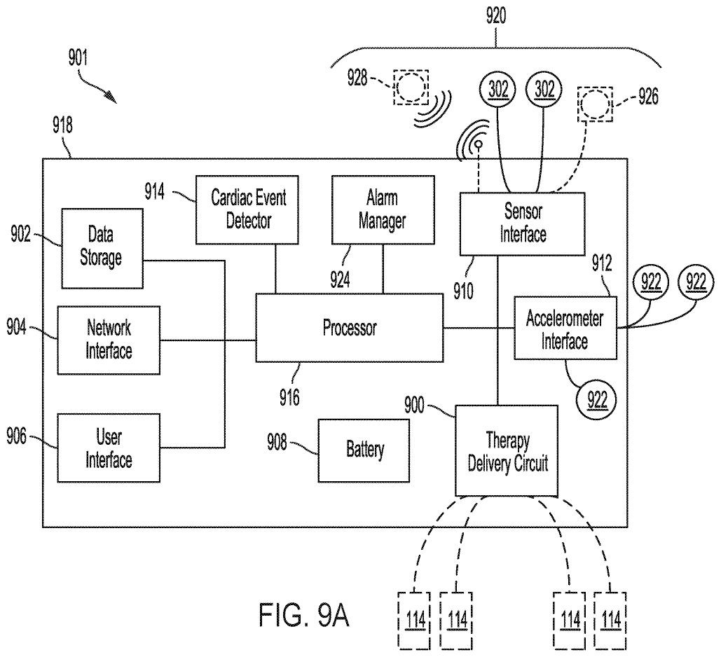

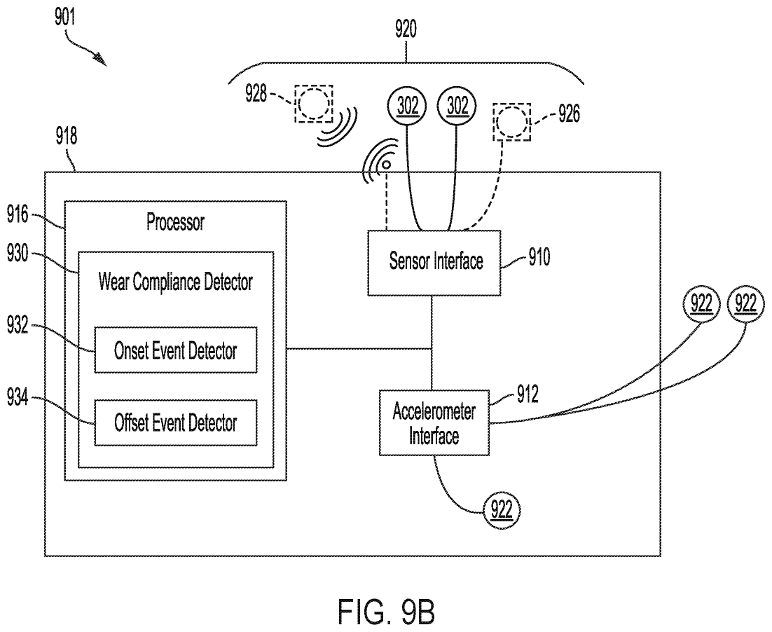

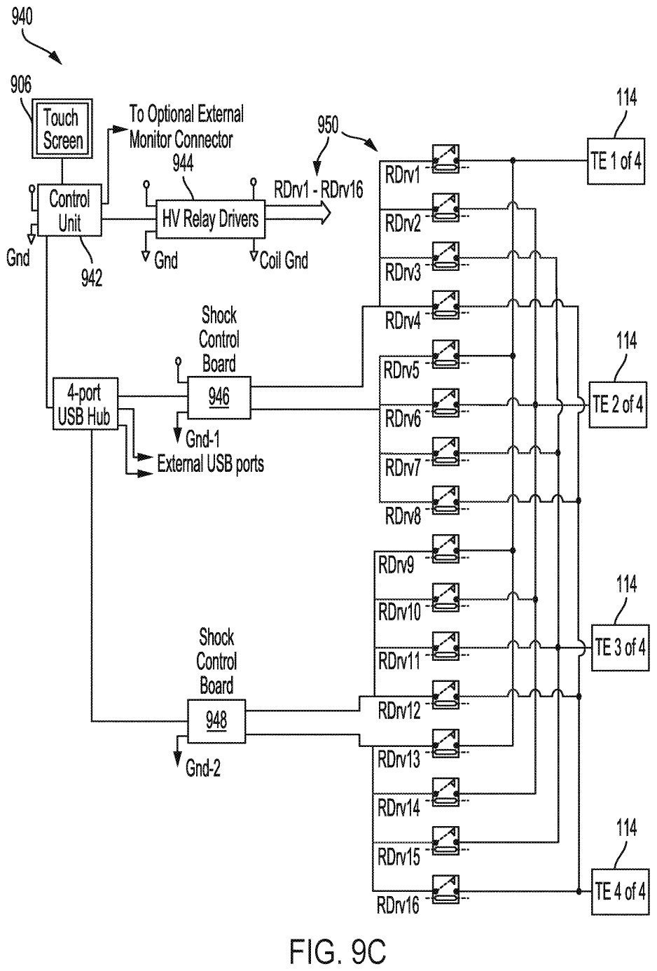

Returning to the wearable defibrillator 100, FIG. 9A illustrates a sample component-level view of a cardiac controller 901 included in a wearable defibrillator 100. The cardiac controller 901 is an example of the cardiac controller 306 shown in FIG. 3 and described above. As shown in FIG. 9A, the cardiac controller 901 can include a housing 918 configured to house a therapy delivery circuit 900 configured to provide one or more therapeutic shocks to the patient 104 via the therapy electrodes 114, a data storage 902, a network interface 904, a user interface 906, at least one battery 908 (e.g., within a battery chamber configured for such purpose), a sensor interface 910 (e.g., to interface with the ECG sensing electrodes 302 and other physiological sensors or detectors such as vibrational sensors, lung fluid sensors, infrared and near-infrared-based pulse oxygen sensors, and blood pressure sensors, among others), a cardiac event detector 914, an alarm manager 924, and at least one processor 916 (e.g., implemented in processing circuitry). As described above, in some implementations, the wearable defibrillator 100 that includes like components as those described above but does not include the therapy delivery circuit 900 and the therapy electrodes 114 (shown in dotted lines). That is, in some implementations, the wearable defibrillator 100 can include the ECG monitoring components and not provide therapy to the patient.

The therapy delivery circuit 900 can be coupled to the therapy electrodes 114 configured to provide therapy to the patient 104. For example, the therapy delivery circuit 900 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit 900 and under the control of one or more processors (e.g., processor 916) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation. In implementations, the therapy delivery circuit 900 is also configured to deliver the cardiac rhythm disruptive shocks (e.g., defibrillation-like shocks, pacing pulses, etc.) discussed above.

In implementations, the therapy delivery circuit 900 includes a first high-voltage circuit connecting a first pair of the therapy electrodes 114 and a second high-voltage circuit connecting a second pair of the therapy electrodes 114 such that the first biphasic therapeutic pulse is delivered via the first high-voltage circuit and the second biphasic therapeutic pulse is delivered via the second high-voltage circuit. In implementations, the second high-voltage circuit is configured to be electrically isolated from the first high-voltage circuit. In implementations, the therapy delivery circuit 900 includes a capacitor configured to be selectively connected to the first high-voltage circuit and/or the second high-voltage circuit. As such, the first high-voltage circuit may powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 900 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 900 can be configured to perform the switching and pulse delivery operations, e.g., under control of the processor 916. As the energy is delivered to the patient 104, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 900 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 902 can include one or more of non-transitory computer-readable media, such as flash memory, solid state memory, magnetic memory, optical memory, cache memory, combinations thereof, and others. The data storage 902 can be configured to store executable instructions and data used for operation of the cardiac controller 901. In some implementations, the data storage 902 can include sequences of executable instructions that, when executed, are configured to cause the processor 916 to perform one or more functions. For example, the data storage 902 can be configured to store information such as ECG data as received from, for instance, the sensor interface 910.

In some examples, the network interface 904 can facilitate the communication of information between the cardiac controller 306 and one or more devices or entities over a communications network. For example, the network interface 904 can be configured to communicate with the remote server 102 or other similar computing device. The network interface 904 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the wearable defibrillator 100). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard. In some implementations, the network interface 904 may be configured to instead communicate directly with the remote server 102 without the use of intermediary device(s). In such implementations, the network interface 904 may use any of the communications links and/or protocols provided above.

In some implementations, the user interface 906 may include one or more physical interface devices, such as input devices, output devices, and combination input/output devices, and a software stack configured to drive operation of the devices. These user interface elements may render visual, audio, and/or tactile content. Thus, the user interface 906 may receive inputs and/or provide outputs, thereby enabling a user to interact with the cardiac controller 306.

The cardiac controller 306 can also include at least one battery 908 configured to provide power to one or more components integrated in the cardiac controller 306. The battery 908 can include a rechargeable multi-cell battery pack. In one example implementation, the battery 908 can include three or more cells (e.g., 2200 mA lithium ion cells) that provide electrical power to the other device components within the cardiac controller 306. For example, the battery 908 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the cardiac controller 306.

The sensor interface 910 can include physiological signal circuitry that is coupled to one or more externally worn sensors configured to monitor one or more physiological parameters of the patient and output one or more physiological signals. As shown, the sensors may be coupled to the cardiac controller 901 via a wired or wireless connection. The sensors can include one or more ECG sensing electrodes 302 (e.g., ECG electrodes) configured to output at least one ECG signal. In some implementations, the sensors can include conventional ECG sensing electrodes and/or digital sensing electrodes. The sensors can also include one or more non-ECG physiological sensors 920 such as one or more vibration sensors 926, tissue fluid monitors 928 (e.g., based on ultra-wide band RF devices), one or more motion sensors (e.g., accelerometers, gyroscopes, and/or magnetometers), a temperature sensor, a pressure sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The one or more vibration sensors 926 can be configured to detect cardiac or pulmonary vibration information. For example, the vibration sensors 926 can detect a patient's heart valve vibration information. For example, the vibration sensors 926 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 926 can also be configured to detect heart wall motion, for instance, by placement of the sensor in the region of the apical beat. The vibration sensors 926 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 926 can also include a multi-channel accelerometer, for example, a three-channel accelerometer configured to sense movement in each of three orthogonal axes such that patient movement/body position can be detected and correlated to detected cardio-vibrations information. The vibration sensors 926 can transmit information descriptive of the cardio-vibrations information to the sensor interface 910 for subsequent analysis.

The tissue fluid monitors 928 can use RF based techniques to assess fluid levels and accumulation in a patient's body tissue. For example, the tissue fluid monitors 928 can be configured to measure fluid content in the lungs, typically for diagnosis and follow-up of pulmonary edema or lung congestion in heart failure patients. The tissue fluid monitors 928 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 928 can transmit information descriptive of the tissue fluid levels to the sensor interface 910 for subsequent analysis.

The controller 901 can further include a motion detector interface operably coupled to one or more motion detectors configured to generate motion data, for example, indicative of physical activity performed by the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity, accelerometer counts indicative of respiration rate, and posture information for the patient 104. For instance, in some implementations, the controller 901 can include an accelerometer interface 912 operably coupled to one or more accelerometers 922, as shown in FIG. 9A. Alternatively, in some implementations, the accelerometer interface 912 may be incorporated into other components of the controller 901. As an example, the accelerometer interface 912 may be part of the sensor interface 910, and the one or more accelerometers 922 may be part of the non-ECG physiological sensors 920.

The accelerometer interface 912 is configured to receive one or more outputs from the accelerometers. The accelerometer interface 912 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 912 can be configured to filter the signals using a high-pass or band-pass filter to isolate the acceleration of the patient due to movement from the component of the acceleration due to gravity.

Additionally, the accelerometer interface 912 can configure the output for further processing. For example, the accelerometer interface 912 can be configured to arrange the output of an individual accelerometer 922 as a vector expressing the acceleration components of the x-axis, the y-axis, and the z-axis as received from each accelerometer. The accelerometer interface 912 can be operably coupled to the processor 916 and configured to transfer the output signals from the accelerometers 922 to the processor for further processing and analysis.

The one or more accelerometers 922 can be integrated into one or more components of the wearable defibrillator 100. In some implementations, one or more motion detectors 922 may be located in or near the ECG sensing electrodes 302. In some implementations, the one or more motion detectors 922 may be located elsewhere on the wearable defibrillator 100. For example, a motion detector 922 can be integrated into the controller 901. In some examples, a motion detector 922 can be integrated into one or more of a therapy electrode 114, an ECG sensing electrode 302, the connection pod 308, and/or into other components of the wearable defibrillator 100. In some examples, a motion detector 922 can be integrated into an adhesive ECG sensing and/or therapy electrode patch.

As described above, the sensor interface 910 and the accelerometer interface 912 can be coupled to any one or combination of sensing electrodes/other sensors to receive patient data indicative of patient parameters. Once data from the sensors has been received by the sensor interface 910 and/or the accelerometer interface 912, the data can be directed by the processor 916 to an appropriate component within the cardiac controller 901. For example, ECG signals collected by the ECG sensing electrodes 302 may be transmitted to the sensor interface 910, and the sensor interface 910 can transmit the ECG signals to the processor 916, which, in turn, relays the data to the cardiac event detector 914. The sensor data can also be stored in the data storage 902 and/or transmitted to the remote server 102 via the network interface 904. For instance, the processor 916 may transfer the ECG signals from the ECG sensing electrodes 302 and the motion data from the one or more accelerometers 922 to the remote server 102.

In implementations, the cardiac event detector 914 can be configured to monitor the patient's ECG signal for an occurrence of a cardiac event such as an arrhythmia or other similar cardiac event. The cardiac event detector can be configured to operate in concert with the processor 916 to execute one or more methods that process received ECG signals from, for example, the ECG sensing electrodes 302 and determine the likelihood that a patient is experiencing a cardiac event, such as a treatable arrhythmia. The cardiac event detector 914 can be implemented using hardware or a combination of hardware and software. For instance, in some examples, cardiac event detector 914 can be implemented as a software component that is stored within the data storage 902 and executed by the processor 916. In this example, the instructions included in the cardiac event detector 914 can cause the processor 916 to perform one or more methods for analyzing a received ECG signal to determine whether an adverse cardiac event is occurring, such as a treatable arrhythmia. In other examples, the cardiac event detector 914 can be an application-specific integrated circuit (ASIC) that is coupled to the processor 916 and configured to monitor ECG signals for adverse cardiac event occurrences. Thus, examples of the cardiac event detector 914 are not limited to a particular hardware or software implementation.

In response to the cardiac event detector 914 determining that the patient 104 is experiencing a treatable arrhythmia, the processor 916 is configured to deliver a cardioversion/defibrillation shock to the patient 104 via the therapy electrodes 114. In some implementations, the alarm manager 924 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). For example, when the processor 916 determines using data from the ECG sensing electrodes 302 that the patient is experiencing a treatable arrhythmia, the alarm manager 924 may issue an alarm via the user interface 906 that the patient is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient that the patient can abort the delivery of the defibrillating shock by interacting with the user interface 906. For instance, the patient may be able to press a user response button or user response buttons on the user interface 906, after which the alarm manager 924 will cease issuing an alert and the cardiac controller 306 will no longer prepare to deliver the defibrillating shock.

In implementations, the cardiac event detector 914 is configured to detect when the patient 104 is experiencing a cardiac rhythm change (e.g., an episode of VF, an episode of VT, a premature ventricular contraction) in response to a cardiac rhythm disruptive shock (e.g., coordinated by the therapy delivery circuit 900) delivered during a baselining session, as discussed above. Depending on the type of cardiac rhythm change, the processor 916 is configured to deliver a cardioversion/defibrillation shock to the patient 104 via the therapy electrodes 114, as discussed above, to restore the patient's normal cardiac rhythm. For example, if the cardiac rhythm change is VF, the processor 916 is configured to deliver a cardioversion/defibrillation shock to the patient 104. The processor 916 is also configured to record, in the data storage 902, data related to the cardiac rhythm change and the cardiac rhythm disruptive shock, as further discussed above (e.g., the energy level of the cardiac rhythm disruptive shock that induced the cardiac rhythm change).

The alarm manager 924 can be implemented using hardware or a combination of hardware and software. For instance, in some examples, the alarm manager 924 can be implemented as a software component that is stored within the data storage 902 and executed by the processor 916. In this example, the instructions included in the alarm manager 924 can cause the processor 916 to configure alarm profiles and notify intended recipients using the alarm profiles. In other examples, the alarm manager 924 can be an application-specific integrated circuit (ASIC) that is coupled to the processor 916 and configured to manage alarm profiles and notify intended recipients using alarms specified within the alarm profiles. Thus, examples of the alarm manager 924 are not limited to a particular hardware or software implementation.

In some implementations, the processor 916 includes one or more processors (or one or more processor cores) that each are configured to perform a series of instructions that result in the manipulation of data and/or the control of the operation of the other components of the cardiac controller 901. In some implementations, when executing a specific process (e.g., cardiac monitoring), the processor 916 can be configured to make specific logic-based determinations based on input data received. The processor 916 may be further configured to provide one or more outputs that can be used to control or otherwise inform subsequent processing to be carried out by the processor 916 and/or other processors or circuitry with which the processor 916 is communicably coupled. Thus, the processor 916 reacts to a specific input stimulus in a specific way and generates a corresponding output based on that input stimulus. In some example cases, the processor 916 can proceed through a sequence of logical transitions in which various internal register states and/or other bit cell states internal or external to the processor 916 may be set to logic high or logic low.

As referred to herein, the processor 916 can be configured to execute a function where software is stored in a data store (e.g., the data storage 902) coupled to the processor 916, the software being configured to cause the processor 916 to proceed through a sequence of various logic decisions that result in the function being executed. The various components that are described herein as being executable by the processor 916 can be implemented in various forms of specialized hardware, software, or a combination thereof. For example, the processor 916 can be a digital signal processor (DSP) such as a 24-bit DSP processor. As another example, the processor 916 can be a multi-core processor, e.g., having two or more processing cores. As another example, the processor 916 can be an Advanced RISC Machine (ARM) processor, such as a 32-bit ARM processor. The processor 916 can execute an embedded operating system and further execute services provided by the operating system, where these services can be used for file system manipulation, display and audio generation, basic networking, firewalling, data encryption, communications, and/or the like.

As noted above, a wearable cardiac treatment device, such as the wearable defibrillator 100, can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 916 of the controller 901 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

The wearable defibrillator 100 is configured for long-term and/or extended use or wear by, or attachment or connection to, a patient. For example, devices as described herein may be capable of being continuously used or continuously worn by, or attached or connected to a patient, without substantial interruption (e.g., up to 24 hours or beyond, such as for weeks, months, or even years). In some implementations, such devices may be removed for a period of time before use, wear, attachment, or connection to the patient is resumed. As an illustration, devices may be removed to change batteries, carry out technical service, update the device software or firmware, and/or to take a shower or engage in other activities, without departing from the scope of the examples described herein. Such substantially or nearly continuous use or wear as described herein may nonetheless be considered continuous use or wear. Additionally, the wearable defibrillator 100 may be configured to transmit signals and data to the remote server 102 continuously or substantially continuously.

As described herein, and noted above, implementations of the present disclosure include monitoring medical device wear compliance for the patient 104. More specifically, the wear compliance information includes an accurate overview of what portion or percentage of a certain time period the patient has worn the wearable defibrillator 100 and how this compares to the expected wear for the patient 104 as prescribed, for example, by their clinician or other healthcare provider when being prescribed the wearable defibrillator 100. FIG. 9B illustrates an example reduced component-level view of the cardiac controller 901 that includes the processor 916 that is configured to monitor wear compliance information for the patient 104 as described herein. For example, the processor 916 can include wear determination circuitry, such as a wear compliance detector 930 as shown in FIG. 9B. The wear compliance detector 930 may be integrated into the processor 916 as illustrated in FIG. 9B, or the wear compliance detector 930 may be integrated as a separate processing component operably coupled to the processor 916. The wear compliance detector 930 can be implemented as a dedicated microprocessor and associated circuitry disposed on a printed circuit board (PCB) along with other components as described herein. The wear compliance detector 930, when implemented in a dedicated microprocessor or integrated into the processor 916, can be based on a series of processor-readable instructions configured to be executed by the dedicated microprocessor or processor 916. For example, the instructions can be implemented in a programming language such as C, C++, assembly language, machine code, HDL, or VHDL. In examples, the dedicated microprocessor can be an Intel-based microprocessor such as an X86 microprocessor or a Motorola 68020 microprocessor, each of which can use a different set of binary codes and/or instructions for similar functions. In implementations, the dedicated microprocessor or processor 916 can be configured to implement wear onset event detection and wear offset event detection as set forth in FIG. 9B.

As further shown in FIG. 9B, the wear compliance detector 930 can include an onset event detector 932 and an offset event detector 934. As described above, the wear compliance detector 930 can be a dedicated microprocessor and associated circuitry disposed on a PCB along with other components as described herein. In implementations, a first microprocessor can be implemented as the onset event detector 932, and a second microprocessor can be implemented as the offset event detector 934. In some implementations, both the onset event detector 932 and offset event detector 934 can be implemented in the same microprocessor as described above. The onset event detector 932 and/or offset event detector 934, when implemented in a dedicated microprocessor or integrated into the processor 916, can be based on a series of processor-readable instructions configured to be executed by the dedicated microprocessor or processor 916.

As noted above, when a patient puts on the wearable defibrillator 100, a wear onset event can be determined based upon analysis of signals received from one or more of the sensors described herein. For example, based upon monitoring of signals output by the ECG sensing electrodes 302 as well as signals output by the accelerometers 922, the onset event detector 932 can determine an onset event indicative of the patient 104 putting on or otherwise wearing the wearable defibrillator 100. Similarly, the offset event detector 934 can determine an offset event indicative of the patient 104 turning off, removing, or otherwise stopping the wearable defibrillator 100 from monitoring. Based upon the measured onset and offset events, the wear compliance detector 930 and/or the processor 916 can determine wear compliance information (e.g., wear determination) for the patient 104.

FIG. 9C illustrates an example circuit 940 for providing therapy shocks as described herein, e.g., such as defibrillation shocks. For instance, the example circuit 940 may be incorporated as part of the therapy delivery circuit 900 illustrated in FIG. 9A. As shown in FIG. 9C, the example circuit 940 includes a control unit 942 configured to initiate an electrical therapeutic pulse sequence. The control unit 942 is electrically connected to the user interface 906, such as a touch screen as shown in FIG. 9C. The control unit 942 may receive, for example, therapeutic pulse parameters such as defibrillation shock energy levels, waveforms for the defibrillation shocks, number of phases delivered in a defibrillation shock, etc. from a technician, clinician, or other caregiver via the user interface 906. As further illustrated in FIG. 9C, the control unit 942 is also electrically connected to relay drivers 944, which are further connected to a series of relays 950. For example, in FIG. 9C, the relays 950 selectively connect four therapy electrodes 114 to a first shock control board 946 and a second shock control board 948. The control unit 942 is further electrically connected to the first shock control board 946 and the second shock control board 948. Each of the first and second shock control boards 946 and 948 includes high-voltage circuitry configured to generate a defibrillation pulse.

As such, when the control unit 942 initiates the electrical therapeutic pulse sequence, the control unit 942 activates the relay drivers 944, which connect the relays for a first set of therapy electrodes 114 forming a first vector to first shock control board 946 and further connects the relays for a second set of therapy electrodes 114 forming a second vector to the second shock control board 948. For example, the relay drivers 944 may selectively connect the relays RDrv1 through RDrv4 to the first shock control board 946 for a first phase of the first therapeutic pulse and selectively connect the relays RDrv5 through RDrv8 to the first shock control board 946 for a second phase of the first therapeutic pulse (e.g., to reverse the polarity of the second phase compared to the first phase). With reference to FIGS. 15 and 16, for example, the relay drivers 944 may selectively connect the relays RDrv1 through RDrv4 to the first shock control board 946 for a first phase of the first therapeutic pulse 1504 or 1604 (e.g., to deliver the positive portion of the biphasic truncated exponential waveform). The relay drivers 944 may then selectively connect the relays RDrv5 through RDrv8 to the first shock control board 946 for a second phase of the first therapeutic pulse 1504 or 1604 (e.g., to deliver the negative portion of the biphasic truncated exponential waveform).

The relay drivers 944 may also selectively connect the relays RDrv9 through RDrv12 to the second shock control board 948 for a first phase of the second therapeutic pulse and selectively connect the relays RDrv13 through RDrv16 to the second shock control board 948 for a second phase of the second therapeutic pulse. The control unit 942 also activates the first shock control board 946 and, according to the predetermined delay, activates the second shock control board 948. The first shock control board 946 generates and delivers a first therapeutic pulse (e.g., a biphasic therapeutic pulse) to the first set of connected therapy electrodes 114. The second shock control board 948 generates and delivers a second therapeutic pulse (e.g., a biphasic therapeutic pulse) to the second set of connected therapy electrodes 114. With reference to FIGS. 15 and 16, for example, the relay drivers 944 may selectively connect the relays RDrv9 through RDrv12 to the second shock control board 948 for a first phase of the second therapeutic pulse 1506 or 1606 (e.g., to deliver the positive portion of the biphasic truncated exponential waveform). The relay drivers 944 may then selectively connect the relays RDrv13 through RDrv16 to the second shock control board 948 for a second phase of the second therapeutic pulses 1506 or 1606 (e.g., to deliver the negative portion of the biphasic truncated exponential waveform). Additionally, in implementations, the relay drivers 944 may activate the relays RDrv9 through RDrv16 after the delivery of the first therapeutic pulse (e.g., as shown in FIG. 15 with the second therapeutic pulse 1506 being delivered after the first therapeutic pulse 1504). Alternatively, in implementations, the relay drivers 944 may activate the relays RDrv9 through RDrv16 while the first therapeutic pulse is still being delivered (e.g., as shown in FIG. 16 with the second therapeutic pulse 1606 being delivered while the first therapeutic pulse 1604 is delivered).

As a further illustration, with reference to FIG. 13, the therapy electrodes 114 may be therapy electrodes 114vv, 114ww, 114pp, and 114qq. The relay drivers 944 may selectively connect the relays RDrv1 through RDrv8 to the first shock control board 946 to deliver the first therapeutic pulse via one of the vectors A-F, and the relay drivers 944 may selectively connect the relays RDrv9 through RDrv16 to the second shock control board 948 deliver the second therapeutic pulse via another of the vectors A-F. As another illustration, with reference to FIG. 14, the therapy electrodes 114 may be therapy electrodes 114rr, 114ss, 114tt, and 114uu. The relay drivers 944 may selectively connect the relays RDrv1 through RDrv8 to the first shock control board 946 to deliver the first therapeutic pulse via one of the vectors G-M, and the relay drivers 944 may selectively connect the relays RDrv9 through RDrv16 to the second shock control board 948 deliver the second therapeutic pulse via another of the vectors G-M.

The example circuit 940 is an illustration of a circuit used to generate and deliver therapeutic pulses to a patient. Other similar circuitry configurations may be used for other embodiments. For example, in some embodiments, the circuitry may include a third shock control board and additional relays connecting the third shock control board to the therapy electrodes 114 such that the wearable defibrillator 100 may deliver three sequential therapeutic pulses to the patient. As another example, in some embodiments, the wearable defibrillator 100 may be configured to deliver therapeutic pulses with more than two phases, such as triphasic or quadriphasic therapeutic pulses. In such examples, the circuit may include additional relays connecting the shock control boards to the therapeutic electrodes. As another example, in some embodiments, the wearable defibrillator 100 may include more than four therapy electrodes 114. Thus, the circuit may include additional relays connecting the additional therapy electrodes 114 to the shock control boards.

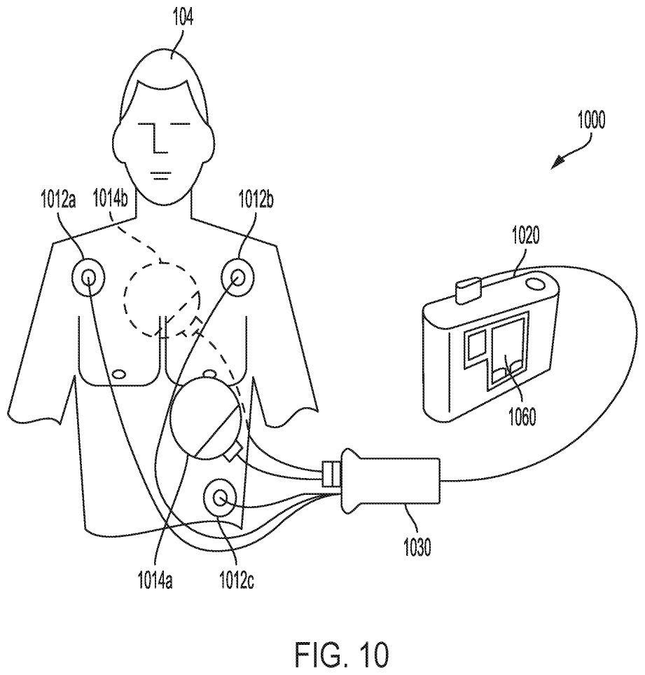

FIG. 10 illustrates another example of a wearable defibrillator 100. More specifically, FIG. 10 shows a hospital wearable defibrillator 1000 that is external, ambulatory, and wearable by the patient 104. Hospital wearable defibrillator 1000 can be configured in some implementations to provide pacing therapy, e.g., to treat bradycardia, tachycardia, and asystole conditions. The hospital wearable defibrillator 1000 can include one or more ECG sensing electrodes 1012a, 1012b, 1012c (e.g., collectively ECG sensing electrodes 1012), therapy electrodes 1014a and 1014b (e.g., collectively therapy electrodes 1014), a medical device controller 1020, and a connection pod 1030. For example, each of these components can be structured and function as similar components of the embodiments of the wearable defibrillator 100 discussed above with reference to FIGS. 3 and 9A-9B. In implementations, the electrodes 1012 and 1014 can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 1014a attaches to the front of the patient's torso to deliver pacing or defibrillating therapy. Similarly, the back adhesively attachable therapy electrode 1014b attaches to the back of the patient's torso. In an example scenario, at least three ECG adhesively attachable sensing electrodes 1012 can be attached to at least above the patient's chest near the right arm (e.g., electrode 1012b), above the patient's chest near the left arm (e.g., electrode 1012a), and towards the bottom of the patient's chest (e.g., electrode 1012c) in a manner prescribed by a trained professional. In implementations, the hospital wearable defibrillator 1000 may include additional adhesive therapy electrodes 1014 and/or the patches shown in FIG. 10 may include additional therapy electrodes 1014 on them such that at least two vectors may be formed between the therapy electrodes 1014 of the hospital wearable defibrillator 1000, as described above.

A patient being monitored by a hospital wearable defibrillator and/or pacing device may be confined to a hospital bed or room for a significant amount of time (e.g., 75% or more of the patient's stay in the hospital). As a result, a user interface 1060 can be configured to interact with a user other than the patient (e.g., a technician, a clinician or other caregiver) for device-related functions such as initial device baselining (e.g., including performing a baselining therapy session), setting and adjusting patient parameters, and changing the device batteries.

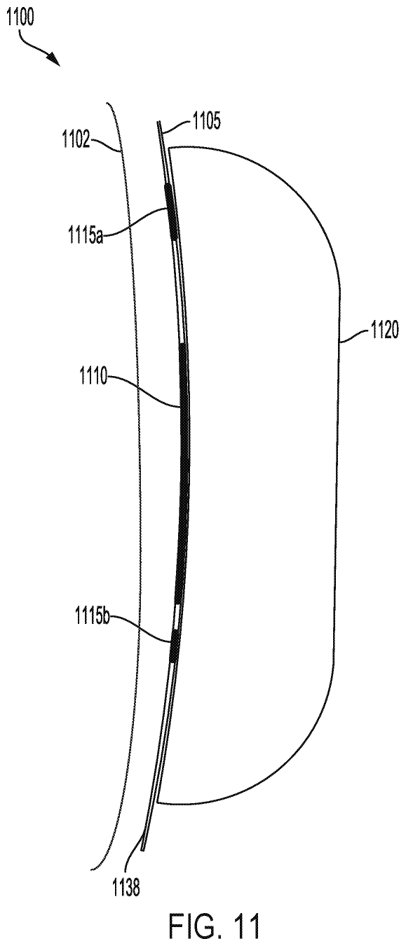

FIG. 11 illustrates another example of a wearable defibrillator 100. As shown in FIG. 11, the wearable defibrillator 100 may be or include an adhesive assembly 1100. The adhesive assembly 1100 includes a contoured pad 1105 and a housing 1120 configured to form a watertight seal with the contoured pad 1105. In implementations, the housing 1120 is configured to house electronic components of the adhesive assembly, such as electronic components similar to components described above with respect to FIGS. 9A and 9B. The adhesive assembly 1100 includes a conductive adhesive layer 1138 configured to adhere the adhesive assembly 1100 to a skin surface 1102 of the patient 104. The adhesive layer 1138 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 1100 also includes at least one of a therapy electrodes 1110 integrated with the contoured pad 1105. In implementations, the adhesive assembly 1100 may include a therapy electrode 1110 that forms a vector with another therapy electrode disposed on another adhesive assembly 1100 adhered to the patient's body and/or with a separate therapy electrode adhered to the patient's body (e.g., similar to therapy electrodes 1014 of FIG. 10). The adhesive assembly 1100 may also include one or more ECG sensing electrodes 1115 integrated with the contoured pad 1105 (e.g., ECG sensing electrodes 1115a and 1115b). In implementations, the adhesive assembly 1100 may alternatively or additionally be in electronic communication with a separate ECG sensing electrode, such as an adhesive sensing electrode adhered to the patient's body. In examples, as shown in FIG. 11, the therapy electrode(s) 1110 and ECG sensing electrode(s) 1115 may be formed within the contoured pad 1105 such that a skin-contacting surface of each component is coplanar with or protrudes from the patient-contacting face of the contoured pad 1105. Examples of a wearable defibrillator 100 including an adhesive assembly 1100 are described in U.S. patent application Ser. No. 16/585,344, entitled “Adhesively Coupled Wearable Medical Device,” filed on Sep. 27, 2019, which is hereby incorporated by reference in its entirety.

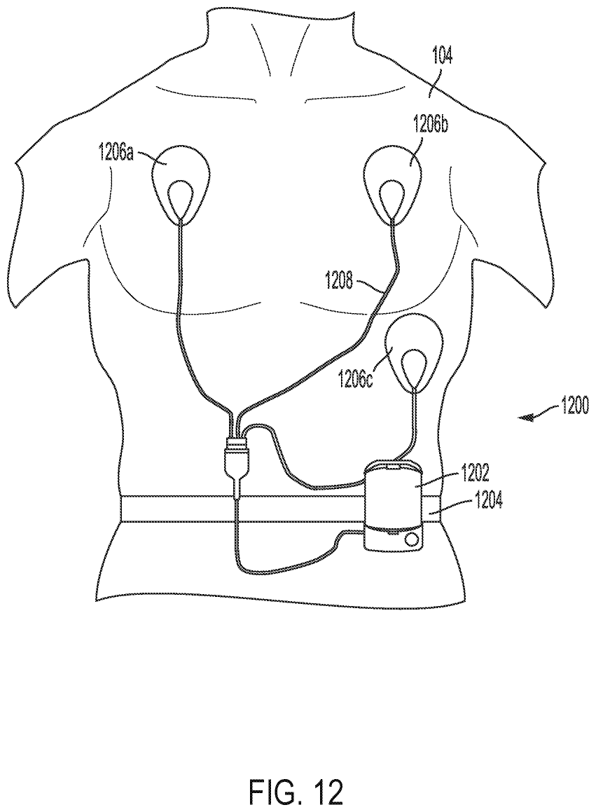

FIG. 12 illustrates another example of a wearable defibrillator 100. As shown in FIG. 12, a wearable defibrillator 100 may include a belted defibrillator 1200 that is external, ambulatory, and wearable by the patient 104. In implementations, the belted defibrillator 1200 may include a medical device controller 1202 configured to be worn mounted on a belt 1204 around the patient's torso. As such, the belted defibrillator 1200 may be configured similarly to the hospital wearable defibrillator 1000 shown in FIG. 10. In implementations, the belted defibrillator 1200 may instead include a medical device controller 1202 integrated into the belt 1204. In such implementations, the belt 1204 includes a number of modules housing the circuitry of the medical device controller 1202 such that the patient 104 does not need to wear a separate medical device controller 1202. Regardless of the implementation, the medical device controller 1202 implemented either as a separate unit or integrated into the belt 1204 is configured to function similarly to the controller described above with reference to FIGS. 3 and 9A-9B.

Similar to the hospital wearable defibrillator 1000, the belted defibrillator 1200 can include adhesive electrodes 1206a, 1206b, 1206c (e.g., collectively adhesive electrodes 1206) configured to be attached to the patient's skin. For example, the adhesive electrodes 1206 may be disposable adhesive electrodes in a wired connection 1208 with the medical device controller 1202 (or, in implementations, with the belt 1204 including the circuitry of the medical device controller 1202). Alternatively, at least some of the adhesive electrodes 1206 may be wirelessly connected to the medical device controller 1202 (or, in implementations, with the belt 1204 including the circuitry of the medical device controller 1202). For instance, the adhesive electrodes 1206 may be configured to communicate via Bluetooth® with the medical device controller 1202 (or the belt 1204). In implementations, at least some of the adhesive electrodes 1206 may include both sensing and therapy components integrated into the same electrode adhesive patch that is attached to the patient. In implementations, at least some of the adhesive electrodes 1206 may be a dedicated sensing electrode or a dedicated therapy electrode. For example, adhesive electrodes 1206a and 1206c may be dedicated therapy electrodes. In implementations, the belted defibrillator 1200 may include additional adhesive electrodes 1206 include sensing and/or therapy components configured to form additional sensing and/or therapy electrode vectors.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-129. (canceled)

130. An ambulatory non-invasive wearable defibrillator for providing therapeutic shocks to restore cardiac function, the ambulatory non-invasive wearable defibrillator comprising:
at least one sensing electrode configured to monitor surface electric signals indicative of cardiac activity of a patient;
a plurality of therapy electrodes;
high-voltage circuitry connecting a first pair of the plurality of therapy electrodes and a second pair of the plurality of therapy electrodes;
a garment configured to be worn about a torso of the patient and further configured to house the at least one sensing electrode, the plurality of therapy electrodes, and the high-voltage circuitry; and
processing circuitry in communication with the at least one sensing electrode and the plurality of therapy electrodes, the processing circuitry being configured to
monitor, using the at least one sensing electrode, the surface electric signals indicative of the cardiac activity of the patient, and
responsive to detecting a suspected cardiac arrhythmia condition based on the cardiac activity,
output an alert for the patient regarding the suspected cardiac arrhythmia condition, and
on expiry of a predetermined response period during which the patient fails to provide a response to the alert, provide an electrical therapeutic pulse sequence to the patient comprising
a first multiphasic therapeutic pulse delivered at a first energy level via a first vector formed by the first pair of therapy electrodes, and
a second multiphasic therapeutic pulse delivered at a second energy level via a second vector formed by the second pair of therapy electrodes,
wherein a timing of the electrical therapeutic pulse sequence comprises a first leading edge of the first multiphasic therapeutic pulse being delivered at a first predetermined time and a second leading edge of the second multiphasic therapeutic pulse being delivered at a second predetermined time following a delay after the first predetermined time.

131. The ambulatory non-invasive wearable defibrillator of claim 130, wherein the first multiphasic therapeutic pulse is delivered at a first higher energy level, and wherein the second multiphasic therapeutic pulse is delivered at a second lower energy level.

132. The ambulatory non-invasive wearable defibrillator of claim 130, wherein one or both of the first multiphasic therapeutic pulse and the second multiphasic therapeutic pulse comprises a truncated exponential waveform.

133. The ambulatory non-invasive wearable defibrillator of claim 130, wherein each of the first multiphasic therapeutic pulse and the second multiphasic therapeutic pulse comprises at least one of a defibrillation shock or a cardioversion shock.

134. The ambulatory non-invasive wearable defibrillator of claim 130, wherein the first multiphasic therapeutic pulse comprises a first biphasic therapeutic pulse, a first triphasic therapeutic pulse, or a first quadriphasic therapeutic pulse; and
wherein the second multiphasic therapeutic pulse comprises a second biphasic therapeutic pulse, a second triphasic therapeutic pulse, or a second quadriphasic therapeutic pulse.

135. The ambulatory non-invasive wearable defibrillator of claim 130, wherein the electrical therapeutic pulse sequence further comprises a third multiphasic therapeutic pulse delivered at a third energy level, and wherein the timing of the electrical therapeutic pulse sequence comprises a third leading edge of the third multiphasic therapeutic pulse being delivered at a third predetermined time following a second delay after the second predetermined time.

136. The ambulatory non-invasive wearable defibrillator of claim 135, wherein the third multiphasic therapeutic pulse is delivered via one of the first vector or the second vector.

137. The ambulatory non-invasive wearable defibrillator of claim 135, wherein the high-voltage circuitry further connects a third pair of the plurality of therapy electrodes, and wherein the third multiphasic therapeutic pulse is delivered via a third vector formed by the third pair of therapy electrodes.

138. The ambulatory non-invasive wearable defibrillator of claim 130, wherein the first pair of therapy electrodes comprises a first therapy electrode configured to be positioned on an anterior portion of the patient's torso and a second therapy electrode configured to be positioned on a posterior portion of the patient's torso; and
wherein the second pair of therapy electrodes comprises a third therapy electrode configured to be positioned on the anterior portion of the patient's torso and a fourth therapy electrode configured to be positioned on the posterior portion of the patient's torso.

139. The ambulatory non-invasive wearable defibrillator of claim 138, wherein the garment comprises two pockets configured to be positioned against the anterior portion of the patient's torso and configured to receive the first and third therapy electrodes, and two pockets configured to be positioned against the posterior portion of the patient's torso configured to receive the second and fourth therapy electrodes.

140. The ambulatory non-invasive wearable defibrillator of claim 130, wherein the first pair of therapy electrodes comprises a first therapy electrode configured to be positioned on a first anterior portion of the patient's torso and a second therapy electrode configured to be positioned on a second anterior portion of the patient's torso, superior to the first therapy electrode; and
wherein the second pair of therapy electrodes comprises a third therapy electrode configured to be positioned on a third anterior portion of the patient's torso and a fourth therapy electrode configured to be positioned on a fourth anterior portion of the patient's torso, superior to the third therapy electrode.

141. The ambulatory non-invasive wearable defibrillator of claim 140, wherein the garment comprises four pockets configured to be positioned against the first, second, third, and fourth anterior positions of the patient's torso and receive the first, second, third, and fourth therapy electrodes.

142. The ambulatory non-invasive wearable defibrillator of claim 130, wherein the first energy level comprises less than 100 J, and wherein the second energy level comprises less than 100 J.

143. The ambulatory non-invasive wearable defibrillator of claim 130, wherein a combined energy level delivered by the first multiphasic therapeutic pulse and the second multiphasic therapeutic pulse comprises less than 100 J.

144. The ambulatory non-invasive wearable defibrillator of claim 130, wherein the first multiphasic therapeutic pulse comprises a waveform lasting between 10 ms and 50 ms, and wherein the second multiphasic therapeutic pulse comprises a waveform lasting between 10 ms and 50 ms.

145. The ambulatory non-invasive wearable defibrillator of claim 130, wherein the delay is between 0 ms and 250 ms.

146. The ambulatory non-invasive wearable defibrillator of claim 130, wherein the processing circuitry is further configured to
receive, via a user interface, a delay user input providing the delay; and
set the delay based on the delay user input.

147. The ambulatory non-invasive wearable defibrillator of claim 130, wherein the delay is greater than a length of the first multiphasic therapeutic pulse.

148. The ambulatory non-invasive wearable defibrillator of claim 130, wherein the delay is less than or equal to a length of the first multiphasic therapeutic pulse.

149. The ambulatory non-invasive wearable defibrillator of claim 130, wherein the high-voltage circuitry comprises
a first high-voltage circuit connecting the first pair of the plurality of therapy electrodes, and a second high-voltage circuit connecting the second pair of the plurality of therapy electrodes;
wherein the first multiphasic therapeutic pulse is delivered via the first high-voltage circuit; and
wherein the second multiphasic therapeutic pulse is delivered via the second high-voltage circuit.
